(19) United States
(12) Patent Application Publication
(10) Pub. No.: US 2024/0139462 A1
(43) Pub. Date: May 2, 2024

(54) METHODS FOR CYBERSICKNESS MITIGATION IN VIRTUAL REALITY EXPERIENCES

(71) Applicant: Rovi Guides, Inc., San Jose, CA (US)

(72) Inventors: Dhananjay Lal, Englewood, CO (US); Reda Harb, Issaquah, WA (US)

(21) Appl. No.: 17/976,517

(22) Filed: Oct. 28, 2022

(51) Int. Cl. A61M 21/00; A63F 13/211; A63F 13/212; A63F 13/65

(52) U.S. Cl. CPC A61M 21/00; A63F 13/211; A63F 13/212; A63F 13/65; A61M 2021/005

(57) ABSTRACT

Systems and methods for mitigating cybersickness that is caused due to display of content, such as a 360° video or a virtual reality experience, are disclosed. The methods measure biometrics of a user to determine a cybersickness score. The score is associated with a cybersickness severity level. A determination is made whether the user's cybersickness severity level exceeds a threshold, and, if so, mitigation or remedial actions are automatically performed. The mitigation options range from altering content, changing device configuration, and automating home automation devices to automating body electronics worn by the user. The type of mitigation option selected is based on the user's cybersickness severity level. The methods also determine demographics of a plurality of users who encountered cybersickness due to engagement with the content. A match between the user's demographics and the plurality of users is determined and accordingly mitigation options are selected on the basis of the match.

### FIELD OF INVENTION

Embodiments of the present disclosure relate to mitigating motion sickness in a virtual reality, motion video, or 360° video setting, by performing content or device level adjustments and using machine learning data to predict motion sickness prior to its occurrence.

### BACKGROUND

Experiencing motion sickness due to playing virtual reality games, experiencing fast-paced motion videos, or navigating through videos that require you to rotate 360° has become common. A term commonly used when referring to such virtual or video-related motion sickness is cybersickness, which is the same as common motion sickness that people may feel in other situations, such as fast car ride, but is based on motion in the cyber world (e.g., videos, virtual experiences, augmented experiences, etc.). Similar to non-cyber motion sickness, cybersickness can also cause nausea, dizziness, headaches, loss of balance, loss of direction, eyesight strain, etc. The type of cybersickness and its severity vary from one person to another, based on each person's tolerance to motion.

Virtual reality (VR) experiences are designed to closely simulate real-life experiences. Developers design VR and other videos such that the user may experience the same effect, sensation, thrill, and motion as in the real world. For example, if the virtual experience relates to a roller coaster, then the VR experience will be designed for the user to feel as if they are on a real-world roller coaster, including feeling all twisting and turning. Since the virtual world is not limited to principles of gravity and physics, the developers can make the experience even more drastic and exhilarating such that the user can feel the sensations that they may not typically experience in the real world. For example, the roller coaster can make drastic drops that are not practical in the real world, or even be situated on another planet or at a 50,000-foot altitude. Such simulations can cause a higher level of sickness than a person may have experienced on a real-world roller coaster.

Current methods provide some solutions to battling cybersickness. These solutions include reducing screen time, taking frequent breaks, focusing away from the screen. All such methods require the user to take a break from watching the video or engaging with a virtual reality experience, to allow the user adequate time to regain composure before attempting to continue watching the video or engaging with a virtual reality experience.

A problem with such solutions is that they require the user to stop watching the video or engaging with a virtual reality experience, rather than providing a solution where cybersickness can be mitigated while the user continues to watch the video or engage with the virtual reality experience.

Another problem with such solutions is that they are left to the user's discretion on when to take a break. Since users are usually intensely involved by the video or virtual reality experience, they either don't take a proactive break at the right time or simply act too late, i.e., only after they are already experiencing cybersickness.

Yet another problem with the current solutions is that they do not provide a customized approach. Since each user has a different level of tolerance, cybersickness may occur for different users based on different types of content, and its severity may vary greatly. Since feedback means are lacking, gathering accurate, complete, and granular data relating to what causes cybersickness and it varies among users has become a challenge. Currently, content developers are not able to fully address motion sickness problems or determine why and how a user may become motion-sick at specific portions of the content.

As such, there is a need for better systems and methods for determining cybersickness and its severity, predicting cybersickness, and automatically mitigating cybersickness while allowing the user to continue enjoying their experience.

### BRIEF DESCRIPTION OF THE DRAWINGS

The various objects and advantages of the disclosure will be apparent upon consideration of the following detailed description, taken in conjunction with the accompanying drawings, in which like reference characters refer to like parts throughout, and in which:

FIG. 1 is a block diagram of a process for mitigating cybersickness in a video or extended reality setting by performing content, device, home automation, or related adjustments, in accordance with some embodiments of the disclosure;

FIG. 2 is a block diagram of an example system for mitigating cybersickness, in accordance with some embodiments of the disclosure;

FIG. 3 is a block diagram of an electronic device used for viewing or engaging with a video, in accordance with some embodiments of the disclosure;

FIG. 4 is another block diagram of an example system for mitigating cybersickness, in accordance with some embodiments of the disclosure;

FIG. 5 is a block diagram of an extended reality (XR) device used for viewing or engaging with a video, in accordance with some embodiments of the disclosure;

FIG. 6 is a flowchart of a process for determining cybersickness severity level and mitigating based on the determined severity level, in accordance with some embodiments of the disclosure;

FIG. 7 is a block diagram of examples of physiological inputs, in accordance with some embodiments of the disclosure;

FIG. 8 is a block diagram of various layers of information that may be used to determine cybersickness, in accordance with some embodiments of the disclosure.

FIGS. 9A and 9B are block diagrams of various levels of cybersickness severity, in accordance with some embodiments of the disclosure;

FIG. 10 is a flowchart of a process for configuring an XR camera or display on the XR camera as part of a mitigation option to reduce cybersickness, in accordance with some embodiments of the disclosure;

FIG. 11 is a block diagram of mitigation options, in accordance with some embodiments of the disclosure;

FIG. 12 is a block diagram of a feedback loop for mitigating cybersickness, in accordance with some embodiments of the disclosure;

FIG. 13 is a block diagram of a feedback loop applied to selected segments and groups of frames for mitigating cybersickness, in accordance with some embodiments of the disclosure;

FIG. 14 is a table of different CSS scores of different users based on a same mitigation option applied based on the severity of cybersickness it can cause, in accordance with some embodiments of the disclosure;

FIG. 15 is an example of a series of mitigation options applied to a plurality of users based on the feedback received, in accordance with some embodiments of the disclosure;

FIG. 16 is a table depicting the results of iterative mitigation options applied based on feedback to reduce the cybersickness score, in accordance with some embodiments of the disclosure;

FIG. 17 is an example of a communication process between an operating system and application or content module for mitigating cybersickness, in accordance with some embodiments of the disclosure;

FIG. 18 is another example of a communication process between an operating system and application or content module for mitigating cybersickness, in accordance with some embodiments of the disclosure; and

FIG. 19 is a block diagram of a plurality of metadata tags applied to content that has been determined to cause cybersickness, in accordance with some embodiments of the disclosure.

### DETAILED DESCRIPTION

In accordance with some embodiments disclosed herein, some of the above-mentioned limitations are overcome by collecting biometrical (physiological or biomarker) data measurements while content is displayed on the user's extended reality device, calculating a cybersickness score based on the collected biometric data measurements, and, in response to determining that the cybersickness score exceeds a severity threshold, automatically executing a remedial action or a cybersickness mitigation option that may be selected based on the cybersickness score, to mitigate the cybersickness. Some of the above-mentioned limitations are overcome by generating and storing a metadata tag in association with the portion of the content item that has been determined to cause cybersickness. The tags can be used by content creators to make content changes to reduce cybersickness. They can also be used as a pointer to alert a subsequent user who is approaching the same content that the tagged content can cause cybersickness. Some of the above-mentioned limitations are overcome by determining that a plurality of users have experienced cybersickness while consuming a portion of XR content and identifying, based on user profiles of the plurality of users, at least one common demographic characteristic for the plurality of users. The methods and systems then determine that a user wearing an extended reality (XR) device is about to consume the same portion of the XR content, and, in response to determining that user profile of the user matches the least one common demographic characteristic for the plurality of users, the methods and systems automatically execute a remedial action for the user, which is selected based on a cybersickness score of the plurality of users.

In some embodiments, the methods obtain physiological data of a user. This may be data obtained from devices worn by the user, such as an extended reality headset, smart glasses, a smart watch, smart clothing, a heart monitor, a blood sugar monitor, etc.

Once the physiological data is collected, a cybersickness score may be calculated based on the collected data. The cybersickness score may range from a scale of minor to extreme, 1 to 10, or any other type of predetermined scale. Based on the cybersickness score, a severity level of cybersickness may be determined. For example, the cybersickness severity may range from a low, medium or moderate to a high severity.

In some embodiments, the system may determine a predetermined threshold for cybersickness. The predetermined threshold may be a point above which cybersickness may be higher than a user can tolerate or above a standard baseline. The threshold may also be point above which a user starts to experience more than minimal cybersickness.

If a determination is made that the current severity threshold for the user based on the collected physiological data is above the predetermined threshold, then the system may automatically perform an action. The action may be a cybersickness mitigation or remedial action to reduce the cybersickness severity level of the user such that the level goes below the predetermined threshold.

In some embodiments, the mitigation option used to reduce the cybersickness score may be to alter the content displayed. In other embodiments, the mitigation option may be to change a device configuration. In yet other embodiments, the mitigation option may be to activate home automation, such as automatically activating a home device that is connected via a network to the extended reality headset, in an effort to reduce the cybersickness score of the user. In still other embodiments, the mitigation option may be to activate a device worn on the body of the user to reduce the cybersickness score of the user.

Once the mitigation option is selected, the system may automatically execute the mitigation option in an effort to reduce the severity level of the cybersickness to the user. In some embodiments, the system may determine whether the mitigation option applied was successful in reducing the cybersickness score. In order to do that, the system may once again collect physiological data when the user is engaged with the same content. Such data may also be fed into a machine learning algorithm to increase its ability for recommending mitigation options as well as predicting motion sickness and its severity.

In addition to taking an action, the system may also tag the portion of the content that caused the cybersickness. The tag may include metadata and have information such as the severity of the cybersickness, what caused the cybersickness, and any other details associated with the user that may be relevant to understanding why it caused cybersickness in the user.

In some embodiments, the system may obtain physiological data from a plurality of users who have experienced cybersickness while consuming a portion of the content. The system may then gather background and demographic details from the users and use them to categorize the users into various groups. For example, the system may group them by age, ethnicity, medical history, history of nausea or vertigo based on medications taken, and other characteristics.

The system may then obtain characteristics of the current user and compare them with the characteristics obtained from the plurality of users. In some embodiments, the system may predict cybersickness for the current user when the current user is consuming the same content based on whether such content also caused cybersickness in the plurality of users who share the same characteristics as the user. For example, if users of the same age and ethnicity as the user experienced cybersickness while the content was displayed to them, then the system would determine that the current user who is of the same age and ethnicity is also likely to experience cybersickness. A wide variety of characteristics may be considered, and predictions may be based on such characteristics. A machine learning model may also be used to predict cybersickness based on data from other pluralities of users with similar characteristics.

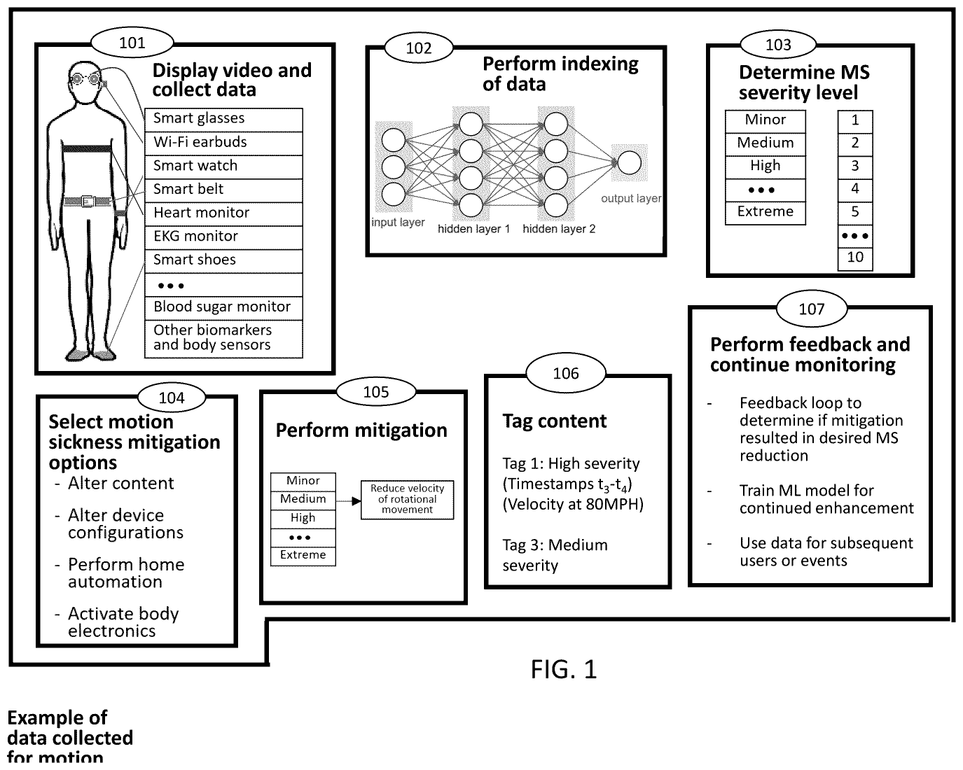

FIG. 1 is a block diagram of a process for mitigating motion sickness in a video setting or extended reality setting by performing content, device, home automation, or related adjustments, in accordance with some embodiments of the disclosure.

In some embodiments, at block 101, a video is displayed on a display device. The video, also referred to herein as content item, may be an extended reality video, a 360° video, a video that displays movements, or any other type of video or virtual display or simulation that causes the user to experience the sensations of a movement.

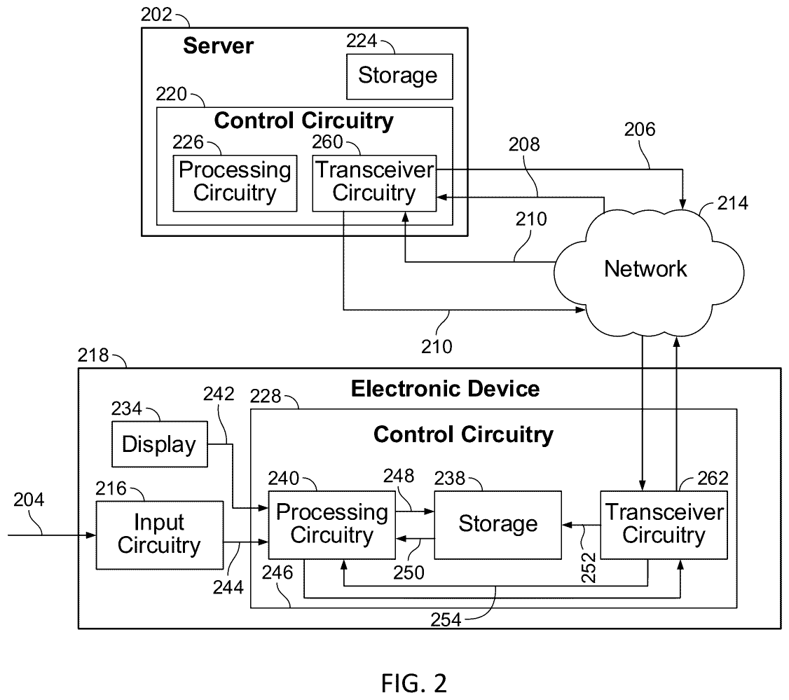

In some embodiments, the video content (also referred to as content, content item, video, virtual reality or VR experience or content, virtual reality video, extended reality or XR content) may be based on extended reality, augmented reality, mixed reality, or any other type of virtual reality, including in the metaverse. In some embodiments, the systems described herein, such as the system in FIGS. 2, 3, and 4, provide an XR environment that includes virtual simulations of both real-world experiences and other experiences that are fictional. For example, the systems may simulate a virtual roller coaster, a virtual game or ride, or a walk through a fictional space, such as a made-up planet with aliens, etc. In other embodiments, the systems may simulate a bungee jump, video game, walking on a rope, playing a movie, playing a game, car ride, or 360° view of the inside a structure, such as a space station. A system may also require or allow the user to perform activities in the virtually simulated environment, such as performing a game maneuver, driving a car on a curve, sitting through a roller coaster ride, or a walking through a virtual layout of a certain city, such as San Francisco or Istanbul.

In some embodiments, these systems utilize an XR device, such as a VR headset, VR glasses, head-mounted display (HMD), or a mobile phone that can act as a VR device, to view or engage with the video content. The XR device may include a display screen for displaying the video content, such as in a virtual reality environment. The XR device may be worn on the user's head such that the display of the XR device is in front of the user's eyes, allowing the user to view the XR 3D simulated environment depicted on the display of the XR device. In some embodiments, when references are made herein to translating or orienting in a 3D space, or viewing, consuming, or engaging with a video, the references are associated with virtual reality devices, such as a virtual reality headset or glasses. In other embodiments, when references are made herein to augmented reality embodiments where a real-world view is used, the references are associated with augmented reality devices, such as an augmented reality headset or glasses through which a real-world environment, XR device, such as a head-mounted display (HMD), as well as virtual overlays on the real-world environment, can be seen via the headset or glasses.

In some embodiments, the XR device may also comprise any one or more of cameras that are facing inward to track the user's gaze, speakers for sound effects, and motion-producing components, such as vibration modules to give the user a sense of feeling effects displayed in the virtual world, such as an earthquake, etc. The XR device may also include accelerometers, gyroscopes, and proximity sensors. It may include a processor, such as a system on a chip (SoC), and memory.

The XR device may be able to connect to the internet and download or access a variety of applications that provide XR experiences to the user, such as a game, a factory setting, a medical operation, training to drive a vehicle, exercising, etc. In some embodiments, the XR device may include more than one connectivity option to communicate with other devices, body sensors, devices that can measure the user's biomarkers (such as heart rate, blood pressure, perspiration, temperature, etc.) or electronic devices for downloading applications. Such connectivity options may include connection via an API, connection using an SoC that features Wi-Fi, Bluetooth, and/or other radio frequency connectivity, in addition to an available USB connection (e.g., USB Type-C).

As mentioned above, the XR device may connect to the internet for downloading videos (content items), such as 360° videos, motion videos, or gaming or other types of applications. These applications may include assets within the virtual world that can move in three-dimensional (3D) space, e.g., in three translational x, y, z axes and three rotational axes, which is commonly referred to as six degrees of freedom (6DOF).

In some embodiments, using the XR device, the systems create a visual for each eye of the user that allows the user to play a virtual game. In some instances, an avatar of a portion of the user's body, an avatar representing their whole body, or no avatar may be used during the virtual game. When an avatar is used, the systems create an illusion that the user is truly in the virtual environment being displayed.

In some embodiments, the XR devices (such as headsets, either augmented reality or virtual reality headsets, or headsets with dual functionality, or virtual glasses, etc.) use head-tracking technology to track the movement of the user's head while they are wearing the device on their head. Such tracking captures the user's head movements as the means of manipulating the camera and viewing things in the virtual world. For example, if the user orients their head to the left, then objects or assets that should be on the left side appear to the user. As such, the visuals change for the user according to how they orient their head, i.e., the XR headset.

In some embodiments, various attachments and accessories that are directly attached to the headset or associated and paired with the headset may be used, such as gaming controllers, biomarker measuring devices, and devices that can obtain the user's physiological input.

In some embodiments, systems may also include wearable devices that can be worn on the body of the user to provide a full-body VR experience. For example, some embodiments may include combining the XR headset with sensors placed on the body of the user to simulate and mimic all body movements performed in the real word as movements on an avatar of the body used in the virtual world. For example, the user may have to physically rotate 270° or 360° or another angle in the real world to simulate the action in the virtual environment, such as in a virtual game. Such rotation in the real world may also be a causing factor of motion sickness in the user as they navigate through the virtual experience.

In some embodiments, the XR environment provided may be stationary, and in other embodiments, the XR environment provided may be dynamically changing, or require a user to rotate or navigate from one location to another. For example, the user may be required to jump from one area to another in the virtual game, or navigate from one virtual room in the XR environment to another room or drive from one location in the XR environment to another location.

In a stationary XR environment, in some embodiments, the user may be able to look around using their XR headset without having to move within the virtual world. For example, stationary experiences may include sitting in a virtual ride, or playing a chess game, which may entail only the limited movement of a sit-and-watch experience.

In some embodiments, other XR experiences may require the user to perform several movements or transitions from one place to another. For example, a user may need to look all around, 360°, to defend from getting hit in a virtual environment where things are thrown at the user from all directions. In such situations, head-tracking is critical and needs to be performed in real time to reduce any lag between the user movements in the real world and the images that should be relayed to the user in the virtual world based on the real-world movements. For example, if the user quickly spins 180° to fight a virtual animal jumping at them from behind, then the images in the virtual world should quickly, with minimal lag, display the animal in motion jumping on the user to provide a real-world-like effect. As such, such dynamically moving experiences are more immersive and require tracking of the headset movements with a higher precision, which may also be a cause that may lead to the user's motion sickness. As referred to herein, motion sickness and cybersickness are used interchangeably and relate to motion sickness in the cyber space, e.g., in extended reality or video viewing, consuming, of an engagement environment.

Referring back to block 101, in some embodiments, data, which is physiological data, may be collected by the control circuitry of a system, such as control circuitries 220 or 228 of the system of FIG. 2. The physiological data, also referred to herein as biomarker data or biomarkers, is data that provides details relating to a user's physical and mental health in real time.

Obtaining physiological readings or biomarkers of the user during the video viewing, consumption, or engagement experience is used by the systems in some embodiments to determine or predict cybersickness and take appropriate mitigation steps (also referred to herein as mitigation options or remedial actions). Such biomarker/physiological input data is important, and research has found significant correlations between motion or cybersickness severity and physiological signals. For example, physiological signals such as gastric dysrhythmia, high eye blink rate, abnormal heart rate, erratic EEG waves and breathing rate are great indicators that the user is experiencing motion or cybersickness.

In some embodiments, physiological readings (also referred to herein as biomarkers) relating to electro-cardiogram (EKG), for example, may also be used as cybersickness indicators. Since different users may have different responses to video content, cybersickness between users may also vary. For example, a study conducted for measuring cybersickness in a VR experience for a set of participants using pre- and post-simulator sickness questionnaires (SSQ) while monitoring the participants using a two-lead ECG varied by user and in some cases showed dramatic differences from one user to the next. The study found significant differences in AVGNN and STDNN of users who report severe cybersickness, thus concluding that there is a correlation between EKG parameters and certain SSQ scores.

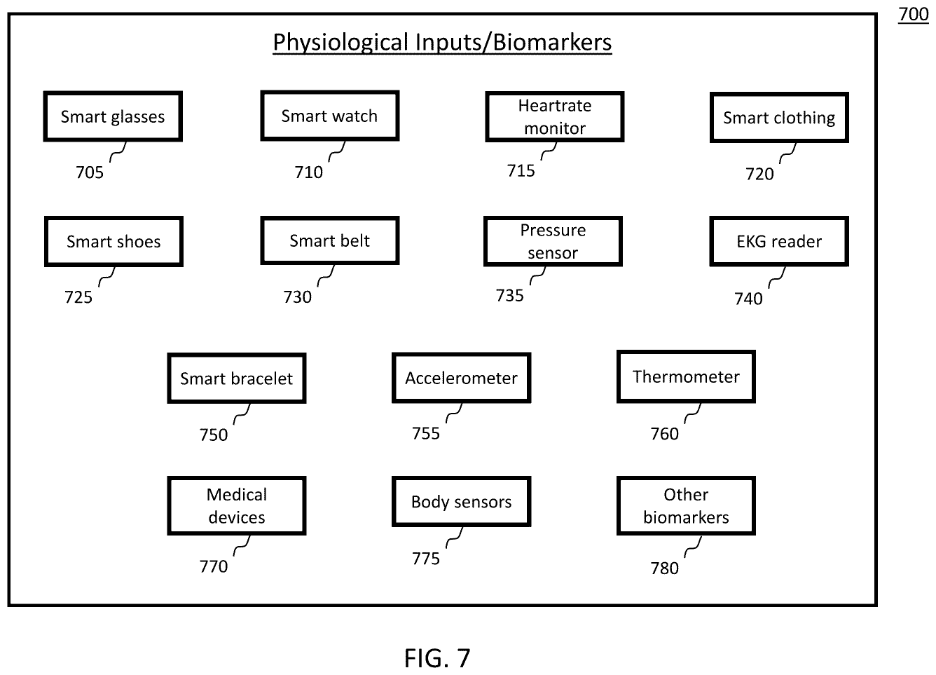

In some embodiments, the biomarkers, such as heart rate, blood oxygen, breathing rate, eye blink rate and other examples of biomarkers shown in FIG. 7, are used by the systems as an indicator of cybersickness. In some embodiments, additional layers of data, such as demographics, medical profile, historical information, and XR device characteristics, such as head-mounted display (HMD) characteristics, are provided to a model, such as a machine learning (ML) model, in non-real time, while biomarkers data that is measured in real time is also provided. ML and AI models used by the system use the data to enhance the predictability of cybersickness as well as determining its severity and the leading causes. ML models also use previous data to predict the likelihood of cybersickness for a current user, and, as more and more data is fed over time, the accuracy of the predictions increases.

Additionally, in some embodiments, the systems and control circuitry may use EEG signals to compute a cybersickness severity (CSS) score. Historical information may be obtained from questionnaire responses (e.g., SSQ or similar for determining susceptibility), historical device measurements (e.g., a smartwatch may provide resting heart rate or the user may allow a health tracking app to periodically share some or all the user's health data with the physiological measurement module), and VR system parameters (e.g., HMD's capabilities such as field of view (FoV), frame rate, resolution, etc.). In some embodiments, the input to the ML model can include medications that have side effects such as dizziness or nausea.

In some embodiments, as depicted in block 101, the biomarker data may be obtained by devices worn by the user, such as smart glasses, Wi-Fi enabled earbuds, smart watch, smart belt, heart monitor, EKG monitor, smart shoes, blood sugar monitor, and other body sensors and devices through which biomarkers can be obtained.

Once the biomarker data is obtained, at block 102, the biomarker data is used as input data. Additional layers of data may be added to further enhance the analysis for improving the predictability of cybersickness.

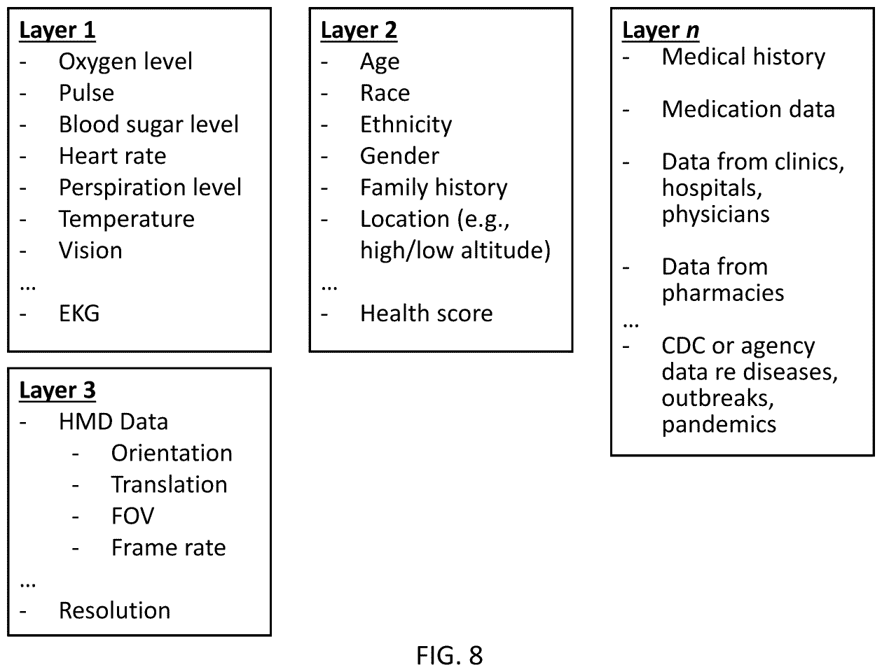

In one embodiment, a first layer of data may include data such as oxygen level, pulse, blood sugar level, heart rate, perspiration level, temperature, vision, and EKG. In another embodiment, a second layer of data may include data relating to age, race, ethnicity, gender, family history, location, and health score. In some embodiments, health score may be a score that in based on a user's overall health and fitness and diet, such as whether the user has any medical conditions, whether the user smokes or drinks, whether the user's cholesterol level is above an acceptable range for their age, the user's weight, exercise regimen etc. In another embodiment, several layers of data may be included. For example, a layer of data may include data from medical history, medical databases, databases from clinics, hospitals and physicians, data from pharmacies, and any government or local agency data such as CDC data relating to disease outbreaks and pandemics. The layers of data are further described in detail in the description of FIG. 8.

In yet another embodiment, a third layer of data may include data from the XR device, such as orientation, translation, field of view, frame rate, and resolution. This layer of data may be obtained from the VR HMD and controllers in real time. The data may be used by the system to account for how movement/activity affects the biomarkers (rather than cybersickness only). It is important to note that different users use different headsets and different auxiliary devices (e.g., smart watches), and therefore, not all of the above physiological parameters can be obtained for all users. As such, layers of data relating to the XR device may also be different for each user.

As depicted at block 103, the layers of data, which are indexed at block 102 into various databases to obtain their cybersickness values, are used to compute an overall cybersickness severity (CSS) score. The scale for a CSS score may be any desired scale created or established by the system or user or may be a predetermined scale. As depicted at block 103, in one environment the scale may vary from minor, medium, or high to extreme severity levels. Based on the CSS score given after computing all the data from 102, one of the severity levels that range from minor to extreme may be associated with the score. For example, a CSS score of 95/100 may be associated with extreme severity, while a score of 15/100 may be associated with a minor severity. The scales for determining severity may be different. For example, as depicted in block 103, a scale from one to 10 may also be used.

The CSS score can also be a score that is assigned to the profile associated with the XR device user. Such score is dynamic, as it changes over time and can be initialized at the beginning of every VR session based on recent biomarker or physiological measurements. It may also be initialized when a determination is made that the user has consumed prescription drugs (e.g., by accessing a health database or if the user logged in to a health app based on a reminder to the user to take their medication).

At block 104, based on the CSS score, one of several mitigation options may be available for selection by the system and a determination may be made of which mitigation option to use. The categories of cybersickness mitigation options include a) altering the content of the video or VR experience, b) altering device configurations, c) performing home automation functions, or d) activating body electronics, i.e., devices that may be worn on the body.

With respect to a), altering the content of the video or VR experience, changes such as altering the content or skipping a portion of the content may be performed. Changes made to the content may be performed to mitigate the user's nausea sensation. These changes may cascade to the application that displays the content, and as such, the system may instruct the application to skip/alter content. Such an alerting or changing action may be taken based on what the user is consuming or experiencing in the video or VR (e.g., the user may be watching streamed content or exploring VR environments such as Horizon Worlds™).

In some embodiments, the change in content may be to reduce the acceleration or velocity of translational or rotational movement in the experience. Such changes may be implemented by using video processing techniques like frame interpolation, if navigation is not user-controllable. The changes may also be performed directly if the acceleration or velocity of translational or rotational movement in the experience is user-controllable.

In another embodiment, a change that may be performed to mitigate cybersickness, i.e., under the alter-content category, may be to add a frame reference, such as a stationary object, to the scene. Such stationary object may provide a non-motion or nonmoving feeling to the user, thereby reducing cybersickness that was caused due to movement of the objects.

In another embodiment, another change that may be performed to mitigate cybersickness under the alter-content category may be to reduce feature complexity of a scene by removing objects. For example, fewer objects may provide clarity in the scene to the user and not cause cybersickness, which may have been caused due to relative motion between the objects.

With respect to b), altering device configurations, in one embodiment, the system may pause or suspend the application and switch to camera pass-through mode. This alteration may allow the user to rebalance and recover from an episode of cybersickness.

In another embodiment, with respect to c) performing home automation functions, a change that may be performed to mitigate cybersickness may be to play calming music, turn on fans or air conditioning in the room, or make upright a reclined chair that is used during the video experience.

In another embodiment, with respect to d) activating body electronics, a change that may be performed to mitigate cybersickness may be to turn on devices attached to the user's body to perform functions that would calm the person, reduce heart rate, cool their body, etc. For example, a device may send pulses or vibrations to the user and the pulses or vibrations caused by the device may calm the user.

Other mitigation options, such as displaying reminders that this environment is not real, switching to a system-guided controlled breathing experience, or using any industry-provided third-party operations that reduce cybersickness may also be automatically executed by the system.

At block 105, in one embodiment, a mitigation option is performed based on a determination made at block 104 that the current cybersickness severity is at a medium level. Accordingly, the system may reduce the velocity or rotational movement of the content, scaling back the amount of motion and the type of motion displayed in an effort to reduce cybersickness.

At block 106, once a segment, portion, or group of frames of a video or VR experience has been identified to cause a moderate or higher level of cybersickness, such segment, portion, or group of frames may be tagged. The tag may be a content metadata tag from the application. Such tagging may increase the system's confidence in taking some actions or may indicate to the system that the content is designed with some flexibility, and the application may accept a command to alter the content or skip a portion of the content. In one embodiment, the content metadata tag is simply an indicator that the content may be modifiable, rather than indicating propensity to cause nausea. The metadata tag may also be used for future users to warn them, ahead of a segment, portion, or group of frames, to expect cybersickness. The tag may also be used for collecting data from other users when they navigate through the tagged portion to determine whether they experience cybersickness and if so, to what level. Such data may be fed into a machine learning algorithm for enhancing the system's accuracy in predicting cybersickness. The tag may also include additional information regarding the portion it is tagging, such as type of cybersickness, severity of cybersickness, potential cause of the cybersickness, such as a car in the VR experience taking a turn on a sharp curve at a high velocity, etc. Generation of such information for the tag may also be automated and/or customized.

At block 107, a feedback loop may be deployed. Such feedback loop may determine whether a mitigation option that was used achieved an anticipated or a positive result. If the mitigation option did not achieve an anticipated result, then such data may be fed back into the system to try additional mitigation approaches until the cybersickness severity level has gone down. In some embodiments, the feedback loop may be used to train the machine learning model for continued enhancement. The data may be used for subsequent users to predict cybersickness. In other embodiments, several iterations of mitigation options may be applied, each after receiving feedback as to whether an earlier mitigation option achieved the desired result.

In some embodiments, FIG. 1 relates to collecting biomarker data for a user, processing the data through block 102 to determine a cybersickness severity level and executing a cybersickness mitigation option. In other embodiments, data from a plurality of users may also be collected. For example, the system may determine that a plurality of users have experienced cybersickness events while consuming a portion of a video. The video/content item may be an XR video, a 360° video, a video with movements or moving parts, or any other type of video or virtual display or simulation that causes the user to experience a movement.

The system may identify, based on user profiles of the plurality of users, at least one common demographic characteristic for the plurality of users. For example, the common demographic may be their age, ethnicity, location, medical history, medications that they are taking, gender, level of experience with VR environments, other VR experiences that they have engaged with, amount of sleep they typically get, their daily working conditions, the type of environment in which they engage with the VR experience, and any other demographic.

The system may also determine that a current user wearing an XR device is about to consume the portion of the XR content that has caused cybersickness in a plurality of users.

The system may then determine whether the user profile of the user matches the at least one common demographic characteristic for the plurality of users, such as age, background, medical history, location, etc. If a determination is made that the user's profile matches the at least one common demographic characteristic for the plurality of users, then the system may automatically execute a remedial or cybersickness mitigation action selected based on data of the cybersickness events experienced by the plurality of users. The system may also collect physiological data from the plurality of users and categorize the users into different groups based on their backgrounds and physiological data. For example, users within a certain age bracket may be in one category and users with a history of nausea or vertigo may be in another category or a subcategory within the age category. The system may determine that the current user falls within one of the categories and apply mitigation options that have worked with users in that particular category. For example, if a particular cybersickness mitigation option did not work with the user that falls in the higher age bracket, and another mitigation option did work, and it is later determined that the user falls in that higher age category, then the system would automatically use the mitigation option that worked with the higher age bracket group of users.

In some embodiments, the system may obtain characteristics of the current user and compare them with the characteristics obtained from a plurality of users. The system may predict cybersickness for the current user when the current user is consuming the same content as consumed by the plurality of users based on whether such content also caused cybersickness in the plurality of users that share the same characteristics as the user. For example, if users of the same age and ethnicity as the user experienced cybersickness while the content was displayed to them, then the system would determine that the current user who is also of the same age and ethnicity is also likely to experience cybersickness. A wide variety of characteristics may be considered, and predictions may be based on such characteristics.

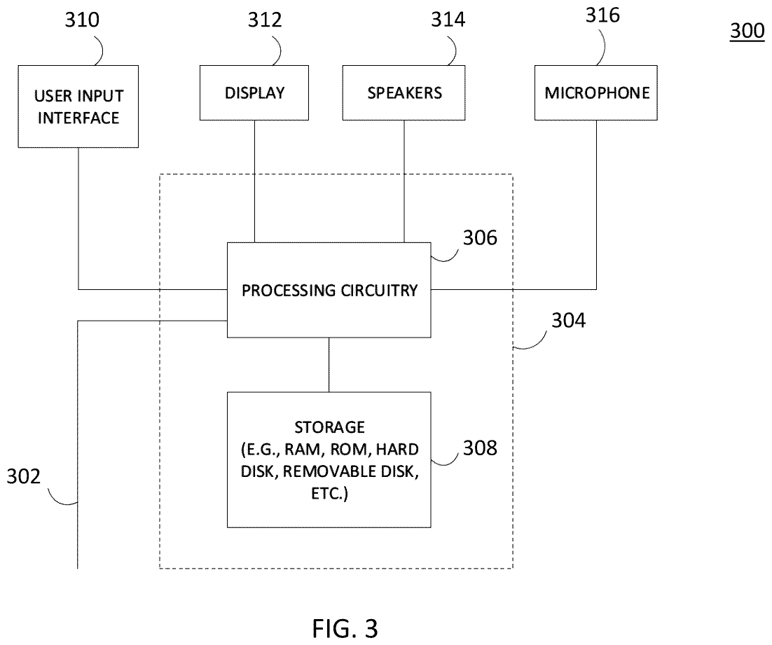

FIG. 2 is a block diagram of an example system for mitigating cybersickness, in accordance with some embodiments of the disclosure and FIG. 3 is a block diagram of an electronic device used for viewing or engaging with a video, in accordance with some embodiments of the disclosure.

FIGS. 2 and 3 also describe example devices, systems, servers, and related hardware that may be used to implement processes, functions, and functionalities described at least in relation to FIGS. 1, 5-19. Further, FIGS. 2 and 3 may also be used for collecting physiological or biomarker data for a single user or a plurality of users, calculating cybersickness scores based on the collected data, associating cybersickness scores with cybersickness severity levels, determining motion sickness or cybersickness mitigation options or remedial actions, automatically performing mitigation actions, tagging content that has been identified to cause cybersickness, training a machine learning model with data collected and the mitigation option applied, determining whether and mitigation option applied was successful in reducing a user's cybersickness score, determining which mitigation option results in a better outcome of reducing cybersickness, automatically executing remedial actions such as performing content changes, changing device configurations, automatically activating home devices, activating home automation functions, activating devices that are worn on a body to reduce cybersickness score, performance feedback operations based on which the system may determine whether a mitigation option was successful and in response perform further mitigation options as necessary, grouping at plurality of users into different groups based on their background, demographics, and other factors, determining whether a current user shares a common characteristic with the plurality of users, applying mitigation options that were applied to the plurality of users if the characteristic between the user and the plurality of users are shared, and performing functions related to all other processes and features described herein.

In some embodiments, one or more parts of, or the entirety of system 200, may be configured as a system implementing various features, processes, functionalities and components of FIGS. 1, and 5-19. Although FIG. 2 shows a certain number of components, in various examples, system 200 may include fewer than the illustrated number of components and/or multiples of one or more of the illustrated number of components.

System 200 is shown to include a computing device 218, a server 202 and a communication network 214. It is understood that while a single instance of a component may be shown and described relative to FIG. 2, additional instances of the component may be employed. For example, server 202 may include, or may be incorporated in, more than one server. Similarly, communication network 214 may include, or may be incorporated in, more than one communication network. Server 202 is shown communicatively coupled to computing device 218 through communication network 214. While not shown in FIG. 2, server 202 may be directly communicatively coupled to computing device 218, for example, in a system absent or bypassing communication network 214.

Communication network 214 may comprise one or more network systems, such as, without limitation, an internet, LAN, WIFI or other network systems suitable for audio processing applications. In some embodiments, system 200 excludes server 202, and functionality that would otherwise be implemented by server 202 is instead implemented by other components of system 200, such as one or more components of communication network 214. In still other embodiments, server 202 works in conjunction with one or more components of communication network 214 to implement certain functionality described herein in a distributed or cooperative manner. Similarly, in some embodiments, system 200 excludes computing device 218, and functionality that would otherwise be implemented by computing device 218 is instead implemented by other components of system 200, such as one or more components of communication network 214 or server 202 or a combination. In still other embodiments, computing device 218 works in conjunction with one or more components of communication network 214 or server 202 to implement certain functionality described herein in a distributed or cooperative manner.

Computing device 218 includes control circuitry 228, display 234 and input circuitry 216. Control circuitry 228 in turn includes transceiver circuitry 262, storage 238 and processing circuitry 240. In some embodiments, computing device 218 or control circuitry 228 may be configured as electronic device 300 of FIG. 3.

Server 202 includes control circuitry 220 and storage 224. Each of storages 224 and 238 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 4D disc recorders, digital video recorders (DVRs, sometimes called personal video recorders, or PVRs), solid state devices, quantum storage devices, gaming consoles, gaming media, or any other suitable fixed or removable storage devices, and/or any combination of the same. Each storage 224, 238 may be used to store various types of content (e.g., videos/content items, 360° videos, extended reality experiences, cybersickness scores, cybersickness severity levels, biometric, physiological or biomarker data related to one or plurality of users, demographics, backgrounds, profiles of a plurality of users, content altering options, device configurations, home automation configurations, body devices configurations, results of mitigation options applied, tags and metadata associated with the tags, virtual reality applications, and AI and ML algorithms). Non-volatile memory may also be used (e.g., to launch a boot-up routine, launch an app, render an app, and other instructions). Cloud-based storage may be used to supplement storages 224, 238 or instead of storages 224, 238. In some embodiments, data relating to videos, 360° videos, extended reality experiences, cybersickness scores, cybersickness severity levels, biometric, physiological or biomarker data related to one or plurality of users, demographics, backgrounds, profiles of a plurality of users, content altering options, device configurations, home automation configurations, body devices configurations, results of mitigation options applied, tags and metadata associated with the tags, virtual reality applications, and AI and ML algorithms, and data relating to all other processes and features described herein, may be recorded and stored in one or more of storages 212, 238.

In some embodiments, control circuitries 220 and/or 228 executes instructions for an application stored in memory (e.g., storage 224 and/or storage 238). Specifically, control circuitries 220 and/or 228 may be instructed by the application to perform the functions discussed herein. In some implementations, any action performed by control circuitries 220 and/or 228 may be based on instructions received from the application. For example, the application may be implemented as software or a set of executable instructions that may be stored in storage 224 and/or 238 and executed by control circuitries 220 and/or 228. In some embodiments, the application may be a client/server application where only a client application resides on computing device 218, and a server application resides on server 202.

The application may be implemented using any suitable architecture. For example, it may be a stand-alone application wholly implemented on computing device 218. In such an approach, instructions for the application are stored locally (e.g., in storage 238), and data for use by the application is downloaded on a periodic basis (e.g., from an out-of-band feed, from an internet resource, or using another suitable approach). Control circuitry 228 may retrieve instructions for the application from storage 238 and process the instructions to perform the functionality described herein. Based on the processed instructions, control circuitry 228 may determine a type of action to perform in response to input received from input circuitry 216 or from communication network 214. For example, in response to obtaining physiological data of a user while the user is displayed portions of the content, the control circuitry 228 may calculate cybersickness scores based on the obtained physiological data. It may also perform steps of processes described in FIGS. 1, 6, 10, 11, 12, 13, and 17-19, including calculating cybersickness scores, departing cybersickness severity level, determining which mitigation options to apply, determining whether the mitigation option applied was successful in reducing the cybersickness score, and predicting cybersickness based on data from plurality of users.

In client/server-based embodiments, control circuitry 228 may include communication circuitry suitable for communicating with an application server (e.g., server 202) or other networks or servers. The instructions for carrying out the functionality described herein may be stored on the application server. Communication circuitry may include a cable modem, an Ethernet card, or a wireless modem for communication with other equipment, or any other suitable communication circuitry. Such communication may involve the internet or any other suitable communication networks or paths (e.g., communication network 214). In another example of a client/server-based application, control circuitry 228 runs a web browser that interprets web pages provided by a remote server (e.g., server 202). For example, the remote server may store the instructions for the application in a storage device. The remote server may process the stored instructions using circuitry (e.g., control circuitry 228) and/or generate displays. Computing device 218 may receive the displays generated by the remote server and may display the content of the displays locally via display 234. This way, the processing of the instructions is performed remotely (e.g., by server 202) while the resulting displays, such as the display windows described elsewhere herein, are provided locally on computing device 218. Computing device 218 may receive inputs from the user via input circuitry 216 and transmit those inputs to the remote server for processing and generating the corresponding displays. Alternatively, computing device 218 may receive inputs from the user via input circuitry 216 and process and display the received inputs locally, by control circuitry 228 and display 234, respectively.

Server 202 and computing device 218 may transmit and receive content and data such as physiological data and cybersickness scores and input from primary devices and secondary devices, such as XR devices. Control circuitry 220, 228 may send and receive commands, requests, and other suitable data through communication network 214 using transceiver circuitry 260, 262, respectively. Control circuitry 220, 228 may communicate directly with each other using transceiver circuits 260, 262, respectively, avoiding communication network 214.

It is understood that computing device 218 is not limited to the embodiments and methods shown and described herein. In nonlimiting examples, computing device 218 may be a primary device, a personal computer (PC), a laptop computer, a tablet computer, a WebTV box, a personal computer television (PC/TV), a PC media server, a PC media center, a handheld computer, a mobile telephone, a smartphone, a virtual, augmented, or mixed reality device, or a device that can perform function in the metaverse, or any other device, computing equipment, or wireless device, and/or combination of the same capable of suitably displaying primary content and secondary content.

Control circuitries 220 and/or 218 may be based on any suitable processing circuitry such as processing circuitry 226 and/or 240, respectively. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors, for example, multiple of the same type of processors (e.g., two Intel Core i9 processors) or multiple different processors (e.g., an Intel Core i7 processor and an Intel Core i9 processor). In some embodiments, control circuitries 220 and/or control circuitry 218 are configured for collecting physiological or biomarker data for a single user or a plurality of users, calculating cybersickness scores based on the collected data, associating cybersickness scores with cybersickness severity levels, determining motion sickness or cybersickness mitigation options or remedial actions, automatically performing mitigation actions, tagging content that has been identified to cause cybersickness, training a machine learning model with data collected and the mitigation option applied, determining whether and mitigation option applied was successful in reducing a user's cybersickness score, determining which mitigation option results in a better outcome of reducing cybersickness, automatically executing remedial actions such as performing content changes, changing device configurations, automatically activating home devices, activating home automation functions, activating devices that are worn on a body to reduce cybersickness score, performance feedback operations based on which the system may determine whether a mitigation option was successful and in response perform further mitigation options as necessary, grouping at plurality of users into different groups based on their background, demographics, and other factors, determining whether a current user shares a common characteristic with the plurality of users, applying mitigation options that were applied to the plurality of users if the characteristic between the user and the plurality of users are shared and performing functions related to all other processes and features described herein, including those described and shown in connection with FIGS. 1, 6, 10, 11, 12, 13, and 17-19.

Computing device 218 receives a user input 204 at input circuitry 216. For example, computing device 218 may receive a user input like motions performed by the user while in an app, movements of the XR headset, and cybersickness experienced by the user while content is displayed.

Transmission of user input 204 to computing device 218 may be accomplished using a wired connection, such as an audio cable, USB cable, ethernet cable or the like attached to a corresponding input port at a local device, or may be accomplished using a wireless connection, such as Bluetooth, WIFI, WiMAX, GSM, UTMS, CDMA, TDMA, 3G, 4G, 4G LTE, or any other suitable wireless transmission protocol. Input circuitry 216 may comprise a physical input port such as a 3.5 mm audio jack, RCA audio jack, USB port, ethernet port, or any other suitable connection for receiving audio over a wired connection or may comprise a wireless receiver configured to receive data via Bluetooth, WIFI, WiMAX, GSM, UTMS, CDMA, TDMA, 3G, 4G, 4G LTE, or other wireless transmission protocols.

Processing circuitry 240 may receive input 204 from input circuit 216. Processing circuitry 240 may convert or translate the received user input 204 that may be in the form of voice input into a microphone, or movement or gestures to digital signals. In some embodiments, input circuit 216 performs the translation to digital signals. In some embodiments, processing circuitry 240 (or processing circuitry 226, as the case may be) carries out disclosed processes and methods. For example, processing circuitry 240 or processing circuitry 226 may perform processes as described in FIGS. 1, 5-19, respectively.

FIG. 3 is a block diagram of an electronic device used for viewing or engaging with a video, in accordance with some embodiments of the disclosure. In an embodiment, the equipment device 300, is the same equipment device 202 of FIG. 2. The equipment device 300 may receive content and data via input/output (I/O) path 302. The I/O path 302 may provide audio content (e.g., such as in the speakers of an XR headset). The control circuitry 304 may be used to send and receive commands, requests, and other suitable data using the I/O path 302. The I/O path 302 may connect the control circuitry 304 (and specifically the processing circuitry 306) to one or more communications paths. I/O functions may be provided by one or more of these communications paths but are shown as a single path in FIG. 3 to avoid overcomplicating the drawing.

The control circuitry 304 may be based on any suitable processing circuitry such as the processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 or i9 processor).

In client-server-based embodiments, the control circuitry 304 may include communications circuitry suitable for allowing communications between two separate user devices to perform the functions of collecting physiological or biomarker data for a single user or a plurality of users, calculating cybersickness scores based on the collected data, associating cybersickness scores with cybersickness severity levels, determining motion sickness or cybersickness mitigation options or remedial actions, automatically performing mitigation actions, tagging content that has been identified to cause cybersickness, training a machine learning model with data collected and the mitigation option applied, determining whether and mitigation option applied was successful in reducing a user's cybersickness score, determining which mitigation option results in a better outcome of reducing cybersickness, automatically executing remedial actions such as performing content changes, changing device configurations, automatically activating home devices, activating home automation functions, activating devices that are worn on a body to reduce cybersickness score, performance feedback operations based on which the system may determine whether a mitigation option was successful and in response perform further mitigation options as necessary, grouping at plurality of users into different groups based on their background, demographics, and other factors, determining whether a current user shares a common characteristic with the plurality of users, applying mitigation options that were applied to the plurality of users if the characteristic between the user and the plurality of users are shared, and performing functions related to all other processes and features described herein.

The instructions for carrying out the above-mentioned functionality may be stored on one or more servers. Communications circuitry may include a cable modem, an integrated service digital network (ISDN) modem, a digital subscriber line (DSL) modem, a telephone modem, ethernet card, or a wireless modem for communications with other equipment, or any other suitable communications circuitry. Such communications may involve the internet or any other suitable communications networks or paths. In addition, communications circuitry may include circuitry that enables peer-to-peer communication of primary equipment devices, or communication of primary equipment devices in locations remote from each other (described in more detail below).

Memory may be an electronic storage device provided as the storage 308 that is part of the control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid-state devices, quantum-storage devices, gaming consoles, gaming media, or any other suitable fixed or removable storage devices, and/or any combination of the same. The storage 308 may be used to store various types of content (e.g., videos, 360° videos, extended reality experiences, cybersickness scores, cybersickness severity levels, biometric, physiological or biomarker data related to one or plurality of users, demographics, backgrounds, profiles of a plurality of users, content altering options, device configurations, home automation configurations, body devices configurations, results of mitigation options applied, tags and metadata associated with the tags, virtual reality applications, and AI and ML algorithms). Cloud-based storage, described in relation to FIG. 3, may be used to supplement the storage 308 or instead of the storage 308.

The control circuitry 304 may include audio generating circuitry and tuning circuitry, such as one or more analog tuners, audio generation circuitry, filters or any other suitable tuning or audio circuits or combinations of such circuits. The control circuitry 304 may also include scaler circuitry for upconverting and down converting content into the preferred output format of the electronic device 300. The control circuitry 304 may also include digital-to-analog converter circuitry and analog-to-digital converter circuitry for converting between digital and analog signals. The tuning and encoding circuitry may be used by the electronic device 300 to receive and to display, to play, or to record content. The circuitry described herein, including, for example, the tuning, audio generating, encoding, decoding, encrypting, decrypting, scaler, and analog/digital circuitry, may be implemented using software running on one or more general purpose or specialized processors. If the storage 308 is provided as a separate device from the electronic device 300, the tuning and encoding circuitry (including multiple tuners) may be associated with the storage 308.

The user may utter instructions to the control circuitry 304, which are received by the microphone 316. The microphone 316 may be any microphone (or microphones) capable of detecting human speech. The microphone 316 is connected to the processing circuitry 306 to transmit detected voice commands and other speech thereto for processing. In some embodiments, voice assistants (e.g., Siri, Alexa, Google Home and similar such voice assistants) receive and process the voice commands and other speech.

The electronic device 300 may include an interface 310. The interface 310 may be any suitable user interface, such as a remote control, mouse, trackball, keypad, keyboard, touch screen, touchpad, stylus input, joystick, or other user input interfaces. A display 312 may be provided as a stand-alone device or integrated with other elements of the electronic device 300. For example, the display 312 may be a touchscreen or touch-sensitive display. In such circumstances, the interface 310 may be integrated with or combined with the microphone 316. When the interface 310 is configured with a screen, such a screen may be one or more monitors, a television, a liquid crystal display (LCD) for a mobile device, active-matrix display, cathode-ray tube display, light-emitting diode display, organic light-emitting diode display, quantum-dot display, or any other suitable equipment for displaying visual images. In some embodiments, the interface 310 may be HDTV-capable. In some embodiments, the display 312 may be a 3D display. The speaker (or speakers) 314 may be provided as integrated with other elements of electronic device 300 or may be a stand-alone unit. In some embodiments, the display 312 may be outputted through speaker 314.

The equipment device 300 of FIG. 3 can be implemented in system 200 of FIG. 2 as primary equipment device 202, but any other type of user equipment suitable for allowing communications between two separate user devices for performing the functions related to implementing machine learning (ML) and artificial intelligence (AI) algorithms, and all the functionalities discussed associated with the figures mentioned in this application.

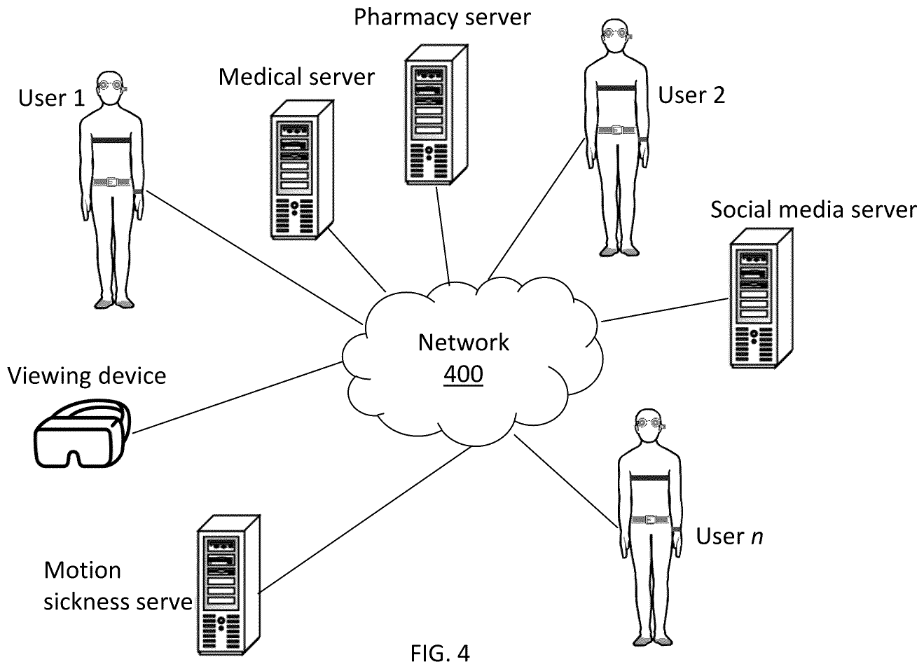

FIG. 4 is another block diagram of an example system for mitigating cybersickness, in accordance with some embodiments of the disclosure. In this embodiment, the system includes a plurality of users, such as user 1, user 2, and user n. The system also includes a viewing device, a motion sickness or cybersickness server, a social media server, a medical server, and a pharmacy server. All the components of the system may be connected to each other via network 400. The network 400 may comprise one or more network systems, such as, without limitation, an internet, the cloud, LAN, Wi-Fi or other network systems suitable for processing applications, accessing and obtaining videos and VR experiences for display, exchanging information, and communicating between system components.

In some embodiments, the system may access a medical server to obtain a medical history of a particular user. The medical history may include the user's medications, clinical history, medical history, and any medical details that may be associated with the user. The system may use such medical information obtained from the medical server as a layer of information that may be fed into a model, such as an ML or AI model, for determining or predicting cybersickness for the particular user.

For example, in one embodiment, the system may obtain physiological input for user 1. The physiological data, in this example, may indicate that, for a current portion of a video, the user should not experience any cybersickness. The system may also obtain medical data for the user from the medical server and add it as a layer of input to further refine the ML and/or AI model. The medical data may indicate that the user has past instances of nausea and vertigo during car rides. Factoring in the medical data, the system may change its prediction, which may have been solely based on physiological input, to now predict that the user is likely to experience cybersickness for the portion of video.

In some embodiments, the system may access a pharmacy server to obtain a medication history of a particular user. The medication and drug history may include the user's current medications, past medications, amounts of medication, and any other medication intake or order fulfillment details associated with the user. The system may use such medication information obtained from the pharmacy server as a layer of information that may be fed into a model, such as an ML or AI model, for predicting cybersickness for the particular user.

For example, in one embodiment, the system may obtain physiological input for user 1. The physiological data, in this example, may be used to predict that based on a current portion of a video the user should not experience any cybersickness. The system may also obtain medication data for the user from the pharmacy server and add it as a layer of input to further refine the ML and/or AI model. The medication information from the pharmacy may indicate that the user takes blood pressure pills, pills to control their blood sugar level due to having diabetes, or pills for a heart condition. Factoring in the medical data, the system may change its prediction, which may have been solely based on physiological input, to now predict that the user is likely to experience cybersickness for the portion of video due to the type of medication taken by the user. The system may also perform a more granular analysis such as that the user has taken their pill within the last hour prior to playing a virtual game. Such medication regimen data may be obtained from the pharmacy server which may include the user's medication intake schedule. Accordingly, the system may alert the user not to play the virtual game if playing so within one hour of taking the medication may cause cybersickness or if it is not advised by their doctor to engage in such activity for at least two to three hours after taking the medication.

In some embodiments, the system may access a social media server to obtain a social media history of a particular user. The social media history may include the user's social activities and posts, including any posts by the user's circle of contacts. For example, the system may access the social media server to determine that the user has gone on a hike that particular morning, i.e., the morning preceding the user's engagement with a 360° video or XR experience. The system may access the social media server and obtain information that indicates that the user ran a marathon, or was sick, or tested positive for Covid-19, or was out drinking late the previous night, or any other information that may be posted by the user or contacts of the user that concerns the user. The system may use such information gathered from the social media server as a layer of information that may be fed into a model, such as an ML or AI model, for predicting cybersickness for the particular user.

For example, in one embodiment, the system may obtain physiological input for user 1. The physiological data, in this example, may indicate that, for a current portion of a video, the user should not experience any cybersickness. The system may also obtain information from the social media server that indicates that the user may had a lot to drink the previous night and was out until 3:00 AM. This may be accessed based on comments posted on a social media site or by determining the timing of the posts. Such information may be added as a layer of input to further refine the ML and/or AI model. Factoring in the information that user was up late, had a lot to drink, and is attempting to play an XR game with varying amounts of rotations, the system may change its determination of the presence of cybersickness, which may have been solely based on physiological input, to now predict that the user is likely to experience cybersickness for the portion of video due to fatigue and hangover.

The motion sickness server may be used to obtain any other data relating to the user and calculate the user's cybersickness score. It may also access various body electronics worn by the user to obtain real-time physiological data that may change dynamically over time. The motion sickness server may also access coordinates of the viewing device and determine the number of rotations and translations that the viewing device has undergone to determine if that amount of movement exceeds the daily or hourly limit and to identify any further motion that may cause cybersickness.

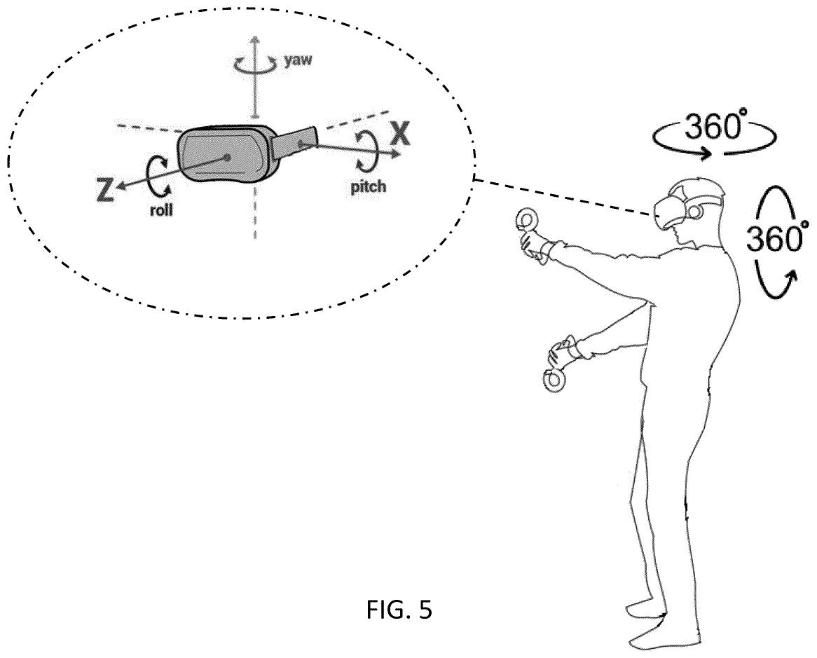

FIG. 5 is a block diagram of an extended reality device used for viewing or engaging with a video, in accordance with some embodiments of the disclosure. The device used to view the video may be an extended reality device, which may include virtual, augmented, or mixed reality headset, smart glasses, or a device that can perform functions in the metaverse, an electronic device, a personal computer (PC), a laptop computer, a tablet computer, a handheld computer, a mobile telephone, a smartphone, or any other device or combination of devices that are suitable for displaying videos or XR content that can be viewed, engaged, or consumed by the user.

In some embodiments, the XR device may include a complete system with a processor and components needed to provide the full XR experience. In other embodiments, the XR device may rely on external devices to perform all the processing, e.g., devices such as smartphones, computers, and servers. For example, the headset may be a plastic, metal, or cardboard holding case that allows viewing, and it may be connected via a wire, wirelessly or via an API to a smartphone and use its screen as lenses for viewing. The headset may also be connected to a gaming console wherein the headset has its own display screen, which is powered by the computer or game console.

As depicted in FIG. 5, in one embodiment, the XR device may be capable of orienting in all 6DOF. Since the headset works by immersing the user into an extended reality environment that has all directions, for a full immersive experience where the user's entire vision, including the peripheral vision, is utilized, an extended reality headset that provides the full 6DOF is preferred (although a extended reality headset with 3DOF can also be used).

Having the 6DOF allows the user to move in all directions and also experience objects and the environment in the virtual world from all directions, e.g., the user can see an object in a 3D space. These 6DOF correspond to rotational movement around the x, y, and z axes, commonly termed pitch, yaw, and roll, as well as translational movement along those axes, which means moving laterally along any one direction x, y, or z. Tracking all 6DOF allows the system to capture the user's movements, as well as their field of view, in both translational as well as rotational directions, thereby providing a full 360° view in all directions.

Although some references have been made to the type of extended reality headset, the embodiments are not so limited, and any other extended reality headset available in the market may also be used with the embodiments described herein. Since XR devices vary, their effects on cybersickness may also vary, and the system may factor such differences in when calculating the cybersickness score. For example, some XR devices may have better resolution or frame rates for display than others, which may have an effect on cybersickness.

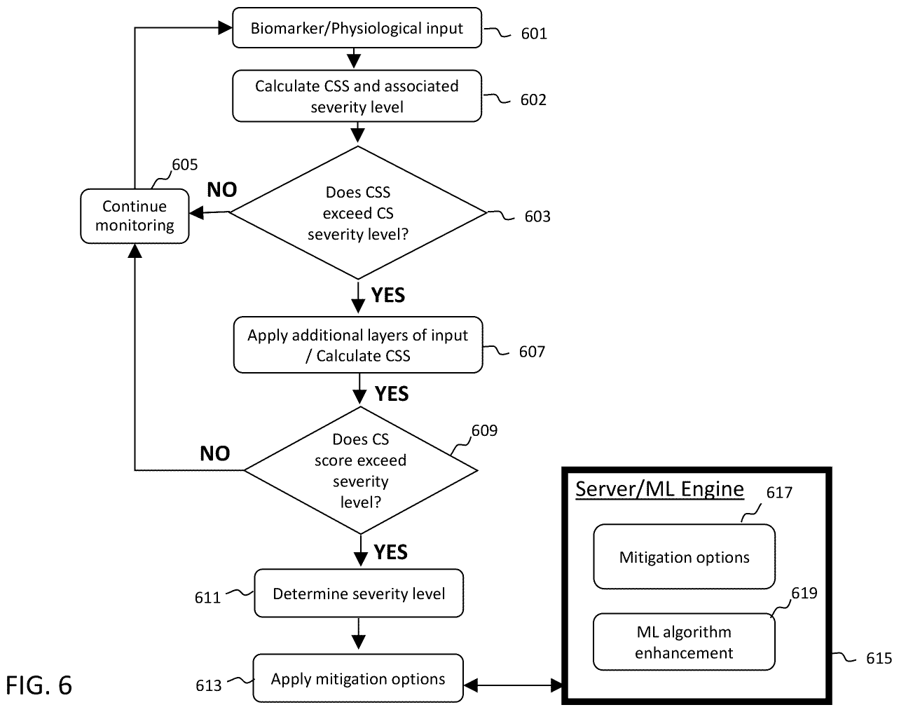

FIG. 6 is flowchart of a process for determining cybersickness severity level and mitigating it based on the determined severity level, in accordance with some embodiments of the disclosure.

In some embodiments, a video is displayed on a display device, such as an XR device. The video/content item may be an extended reality video, a 360° video, a video with motions or moving parts, or any other type of video or virtual display or simulation that causes the user to experience a movement. The video may also be based on extended reality, which may be virtual reality, augmented reality, mixed reality, or any other type of reality, including in the metaverse.

In some embodiments, the user may be wearing one or more electronic devices on their body. For example, the user may be wearing smart glasses, Wi-Fi enabled earbuds, smart watch, smart belt, heart monitor, EKG monitor, smart shoes, blood sugar monitor, and other body sensors and devices through which biomarker input can be obtained. In other embodiments, attachments and accessories that are directly attached to the headset or associated and paired with the headset as well as gaming controllers with sensors and other biomarker measuring devices may also be used.

In some embodiments, the user may also be wearing motion sensors or trackers that emulate the user's movements in a virtual environment. For example, the user may rotate their hand that is wearing a tracker and the hand in the virtual environment may also move in the same manner.

Regardless of which type of wearable devices, attachments to the XR device, or trackers used, the control circuitries 220 or 228, at block 601, may obtain the user's biomarkers through such devices, attachments, and trackers. Such biomarker data may include electrocardiogram (EKG) readings, heart rate, blood oxygen, breathing rate, eye blink rate, body temperature, perspiration level, dizziness, loss of concentration, lack of responsiveness to game challenges, headaches, nausea, yawning, sighing, increased salivation, burping, blurred vision, drowsiness, spatial disorientation or any other biomarker-related readings of the user while the user is viewing, consuming, or engaging with the video.

In one embodiment, the physiological readings or biomarkers obtained at block 601 during the video viewing, consumption, or engagement experience are used by the control circuitries 220 and/or 228 to determine or predict cybersickness. In one embodiment, at block 602, the physiological readings or biomarkers obtained at block 601 are used by the control circuitries 220 and/or 228 to calculate a cybersickness severity (CSS) score and then determine a severity level associated with the score. The severity levels may be predetermined and associated with a range of CSS. For example, 0-25 may be associated with minor severity of cybersickness, 26-50 may be associated with medium severity of cybersickness, 51-75 may be associated with high severity of cybersickness, and 76-100 may be associated with extreme severity of cybersickness. The scale is exemplary, and any other type of scale, such as a severity scale of 1-10 or a rating of A-Z may also be used.

At block 603, control circuitries 220 and/or 228 may determine whether the CSS score exceeds a cybersickness severity level. For example, if the CSS score is associated with a medium, high, or extreme severity level, the control circuitries 220 and/or 228 may determine that the CSS score exceeds a threshold level. The threshold for exceeding the severity level may be predetermined. For example, the threshold may be set at a certain CSS number, such as 50 or 75, or a certain severity level, such as a moderate severity level. If the CSS score exceeds the predetermined severity level, then the control circuitry may determine that cybersickness is likely to occur.

If a determination is made at block 603 that the CSS score does not exceed the cybersickness severity level, i.e., which can be associated with the user not being likely to experience cybersickness, then, at block 605, in one embodiment, the control circuitries 220 and/or 228 may continue monitoring the user's progression through the video or extended reality experience and repeat blocks 601-603.

If a determination is made at block 603 that the CSS score does exceed the cybersickness severity level, then, in one embodiment, the control circuitries 220 and/or 228 may skip blocks 607-611 (not depicted in figure) and apply mitigation options at block 613.

In another embodiment, at block 607, the control circuitries 220 and/or 228 may use additional information as layers of input to refine the model and determine the likelihood of cybersickness occurring in the user. In some embodiments, the additional layers of data, such as demographics, medical profile, historical information, and XR device characteristics may be used as input. In other embodiments, several layers of input may be applied on a sequential basis. For example, a first layer of data may include data such as oxygen level, pulse, blood sugar level, heart rate, perspiration level, temperature, vision comma and EKG. In another embodiment, second layer of data may include data relating to age, race, ethnicity, gender, family history, location, and health score. Additional layers of input data may also include data from medical history; medical databases; databases from clinics, hospitals and physicians; data from pharmacies; and any government or local agency data such as CDC data relating to diseases, outbreaks and pandemics. The layers of data may also include data from the XR device, such as orientation, translation, field of view, frame rate, and resolution. This layer of data may be obtained from the VR HMD and controllers in real time.

The control circuitries 220 and/or 228 may apply any combination of the layers of inputs and recalculate the CSS score. If a determination is made at block 609 that the CSS score does exceed the cybersickness severity level, then, at block 611, in one embodiment, the control circuitries 220 and/or 228 may determine the exact severity level, including a sub-range within each severity level, and apply mitigation options at block 613. The mitigation options may be obtained from the server or ML engine 615. The control circuitries 220 and/or 228 may also store all data relating to CSS scores, at which portion of the video such CSS scores were measured, the mitigation options applied, and the result of the mitigation options. The stored data may be used by the ML engine to further train the ML algorithm such that the algorithm is enhanced based on the volumes of input received over time to increase its cybersickness detection function and to provide more accurate mitigation options that worked for others in the past.

If a determination is made at block 609 that the CSS score does not exceed the cybersickness severity level, i.e., which can be associated with the user not being likely to experience cybersickness, then, at block 605, in one embodiment, the control circuitries 220 and/or 228 may continue monitoring the user's progression through the video or extended reality experience and repeat blocks 601-603.

The control circuitries 220 and/or 228 may also deploy a feedback loop where mitigation options are applied, and the CSS scores are reassessed to determine the effect of the mitigation options. If further mitigation is needed, such as when the severity level is not brought below the expected severity level, then control circuitries 220 and/or 228 may apply different or more of the same mitigation options.

FIG. 7 is a block diagram of examples of physiological input devices, in accordance with some embodiments of the disclosure.

In one embodiment, the user may be wearing a pair of smart glasses 705. The current user may be wearing these while consuming a video. The control circuitries 220 and/or 228 may access the smart glasses 705 to obtain any physiological or biomarker inputs from them. For example, the control circuitries 220 and/or 228 may obtain the user's gaze, dilation in the user's eyes, eye pigmentation (which can indicate fatigue) as compared to a baseline pigmentation obtained earlier, degree of attention, frequency of eyes closing, water content in the eyes as an indicating factor of tiredness, lack of sleep, or other medical condition. Such data may be used to determine whether the user is experiencing cybersickness, and if so at what level, or if the user is likely to experience cybersickness.

In one embodiment, the user may be wearing a smart watch 710. The user may be wearing the smart watch while consuming a video. The control circuitries 220 and/or 228 may access the smart watch 710 to obtain any physiological or biomarker inputs from it. Some smart watches include several sensors, such as 16+ sensors. These sensors may be fitness trackers, movement trackers, altimeters, optical heart rate sensors, blood oxygen sensors, bioimpedance sensors, EKG sensors, gyroscopes, GPS sensors, electrodermal activity sensors, skin temperature sensors and more. For example, the control circuitries 220 and/or 228 may access the sensors to obtain data that may serve as an indicating factor of the user's health, medical condition, tiredness, lack of sleep, fitness and other vitals. Such data may be used to determine whether the user is experiencing cybersickness, and if so at what level, or if the user is likely to experience cybersickness.

In one embodiment, the user may be wearing a heart rate monitor 715. The user may be wearing such a heart rate monitor while consuming a video. The control circuitries 220 and/or 228 may access the heart rate monitor 715 to obtain any physiological or biomarker inputs from it. For example, the control circuitries 220 and/or 228 may obtain heart health data, data relating to unusual rhythms and heart behaviors for further investigation and detect and monitor heart or pulse rate. Such data may be used to determine whether the user is experiencing cybersickness, and if so at what level, or if the user is likely to experience cybersickness.

In one embodiment, the user may be wearing smart clothing 720. The user may be wearing this clothing while consuming a video. The control circuitries 220 and/or 228 may access the smart clothing 720 to obtain any physiological or biomarker inputs from it. For example, the control circuitries 220 and/or 228 may obtain the user's body temperature, heart rate, and perspiration level. Such data may be used to determine whether the user is experiencing cybersickness, and if so at what level, or if the user is likely to experience cybersickness.

In one embodiment, the user may be wearing smart shoes 725. The user may be wearing these smart shoes while consuming a video. The control circuitries 220 and/or 228 may access the smart shoes 725 to obtain any physiological or biomarker inputs from them. For example, the control circuitries 220 and/or 228 may determine the amount of walking or running performed by the user during the day on which they are viewing or consuming the video. They may also determine the walking patterns and if the user is tired or sober after they had much to drink. Such data may be used to determine whether the user is experiencing cybersickness, and if so at what level, or if the user is likely to experience cybersickness.

In one embodiment, the user may be wearing a smart belt 730. The user may be wearing the smart belt while consuming a video. The control circuitries 220 and/or 228 may access the smart belt 730 to obtain any physiological or biomarker inputs from it. For example, the control circuitries 220 and/or 228 may determine whether the user has just consumed a large meal which resulted in tightening of the belt, if their mass has grown, body temperature, etc. Such data may be used to determine whether the user is experiencing cybersickness, and if so at what level, or if the user is likely to experience cybersickness.

In one embodiment, the user may be wearing a device with a pressure sensor 735, or an EKG reader 740. Data from such devices may be used to determine whether the user is experiencing cybersickness, and if so at what level, or if the user is likely to experience cybersickness.

In one embodiment, the user may be wearing a smart bracelet 750. The current user may be wearing the smart bracelet while consuming a video. The control circuitries 220 and/or 228 may access the smart bracelet 750 to obtain any physiological or biomarker inputs from it. For example, the control circuitries 220 and/or 228 may determine the user's pulse at the forearm, their blood sugar level, perspiration level, body temperature, etc. Such data may be used to determine whether the user is experiencing cybersickness, and if so at what level, or if the user is likely to experience cybersickness.

In one embodiment, the user may be wearing a device that includes an accelerometer 755. The user may be wearing the device with the accelerometer while consuming a video. The control circuitries 220 and/or 228 may access the accelerometer 755 to obtain any physiological or biomarker inputs from it. For example, the control circuitries 220 and/or 228 may determine the user's physical activity, such as their speed or acceleration during an exercise or a game. Such data may be used to determine whether the user is experiencing cybersickness, and if so at what level, or if the user is likely to experience cybersickness.

In one embodiment, the user may be wearing a device that includes a thermometer 760, and data from such thermometer, i.e., the user's body temperature, may be used to determine whether the user is experiencing cybersickness, and if so at what level, or if the user is likely to experience cybersickness.

In addition to the listed devices, the user may also be wearing other types of medical devices 770, body sensors 775, and devices through which other biomarker data 780 can be obtained. For example, the user may be wearing a device that includes an oximeter for measuring the user's oxygen levels. Data from all such devices may be used to determine whether the user is experiencing cybersickness, and if so at what level, or if the user is likely to experience cybersickness. Additionally, XR device characteristics may also be provided to the control circuitries to factor them in when calculating cybersickness severity scores.

FIG. 8 is a block diagram of various layers of information that may be used to determine cybersickness, in accordance with some embodiments of the disclosure.

In one embodiment, four layers of data are depicted in FIG. 8. Although four layers are depicted, the embodiments are not so limited and any more or fewer layers of data or just one layer of data with all the types of data are contemplated. As depicted, a first layer of data may include oxygen level, pulse, blood sugar level, heart rate, perspiration level, temperature, vision, and EKG. A second layer of data, as depicted, may include data relating to age, race, ethnicity, gender, family history, location, and health score. A third layer of data may include data from the XR device, such as an HMD, such as orientation, translation, field of view (FOV), frame rate, and resolution. This layer of data may be obtained from the VR HMD and controllers in real time. An nth layer of data may include data from medical history; medication data; databases from clinics, hospitals and physicians; data from pharmacies; and any government or local agency data such as CDC data relating to diseases outbreaks and pandemics.

The layers of data may relate to the current user or a plurality of users. For example, in some embodiments, data from a plurality of users relating to data layer 1, layer 2, layer 3, and layer n may be collected by the control circuitries. The data from such plurality of users may be categorized based on their age, race, heart rate, type of device they use, or any other information from any one of the layers. A current user's data may be compared with data from the plurality of users to determine if a match occurs. For example, if a current user's race, age, gender, or any of the metrics from the layers 1-n is the same as that of the plurality of users, then data from such plurality of users may be deemed more applicable to the current user.

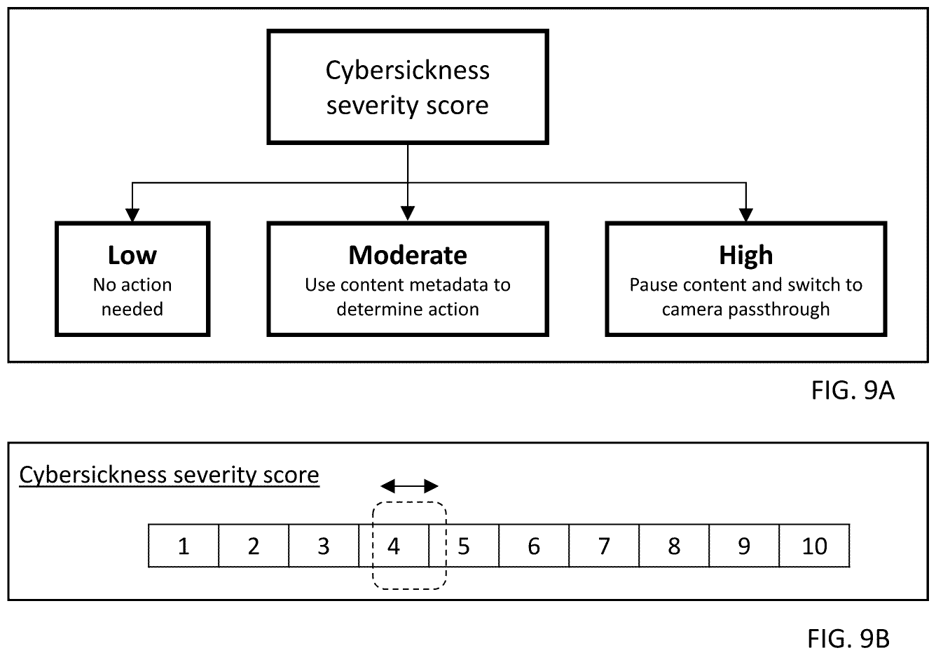

FIGS. 9A and 9B are block diagrams of various cybersickness severities, in accordance with some embodiments of the disclosure.

In one embodiment, the cybersickness severity scale may be a scale that ranges from low to high severity levels. For example, a CSS score of 1-33/100 may be associated with low severity level, a CSS score of 34-66/100 may be associated with moderate severity level, and a CSS score of 67-100/100 may be associated with high severity level. In some embodiments, when a low severity level is detected, the control circuitry may not perform any mitigation action. In other embodiments, when the severity scale is associated with either a moderate or high severity, then the control circuitry may perform mitigation actions associated with the level of severity.

In one embodiment, if the severity scale is associated with moderate severity, then the control circuitry may use content metadata to determine an action such as altering the content, altering a configuration on the device, performing home automation, or activating body electronics to lower the cybersickness severity level in the user.

In another embodiment, if the severity scale is associated with a high severity level, then the control circuitry may either pause the video or switch the XR device from a video viewing mode to a pass-through mode where the real world can be seen through the XR headset.

The scale is exemplary, and any other type of scale, such as a severity scale of 1-10 depicted in FIG. 9B or other scales of, for example, 1-100 or a rating of A-Z may also be used.

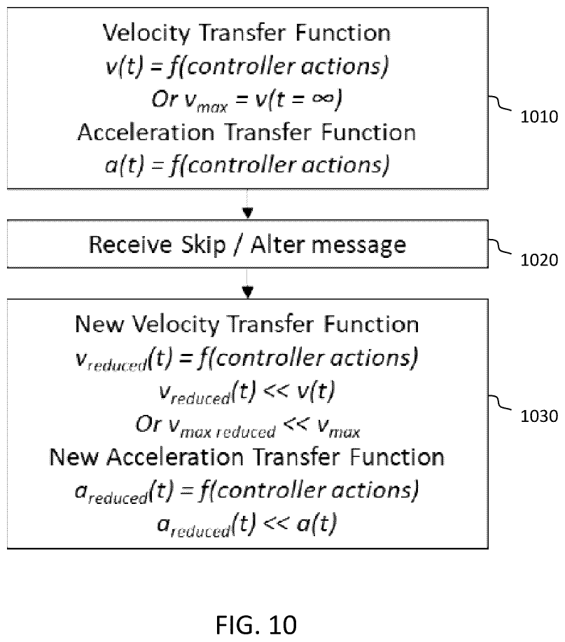

FIG. 10 is flowchart of a process for configuring an XR camera or display on the XR camera as part of a mitigation option to reduce cybersickness, in accordance with some embodiments of the disclosure.

In one embodiment, as depicted in the flowchart of FIG. 10, the camera associated with the XR device to view, consume, or engage with a video is configured based on a calculation. The camera parameters (in a gaming engine SDK) may be configured to limit its acceleration, speed, or speed when a skip/alter message is received in real time. The XR camera object may be attached to the user's XR device to render a first-person view on the display. The velocity and/or acceleration transfer function of the user's 3D avatar (and therefore also the XR camera) is dependent on the locomotion system developed/chosen by the content creator. In one embodiment, the velocity and/or acceleration can be made less sensitive to the controller actions. For example, if the user moves their controller back and forth to navigate in their environment, then the velocity developed per unit of user controller action can be reduced. In another embodiment, the peak or maximum velocity can be limited to/capped at a lower value.

As depicted, the velocity transfer function is used by the control circuitry to transfer from current velocity of acceleration at block 1010 to a reduced velocity and acceleration at block 1030 when the control circuitry receives a skip/alter message 1020. As mentioned earlier, the skip/alter message may be received when the cybersickness severity level is determined to be moderate or higher than moderate but not extreme. In other words, switching the XR camera to pass through such that the real-world environment can be visualized by the user may be reserved for the highest level of cybersickness severity. In other embodiments, different types of ranges may be predetermined for when to reduce velocity and acceleration and when to use the pass-through feature. The ranges may be predetermined based on the user's demographic and physiological inputs and may be customized differently for each user. For example, the threshold for switching to pass-through visualization may be different from one user to another and may depend on their tolerance of cybersickness.

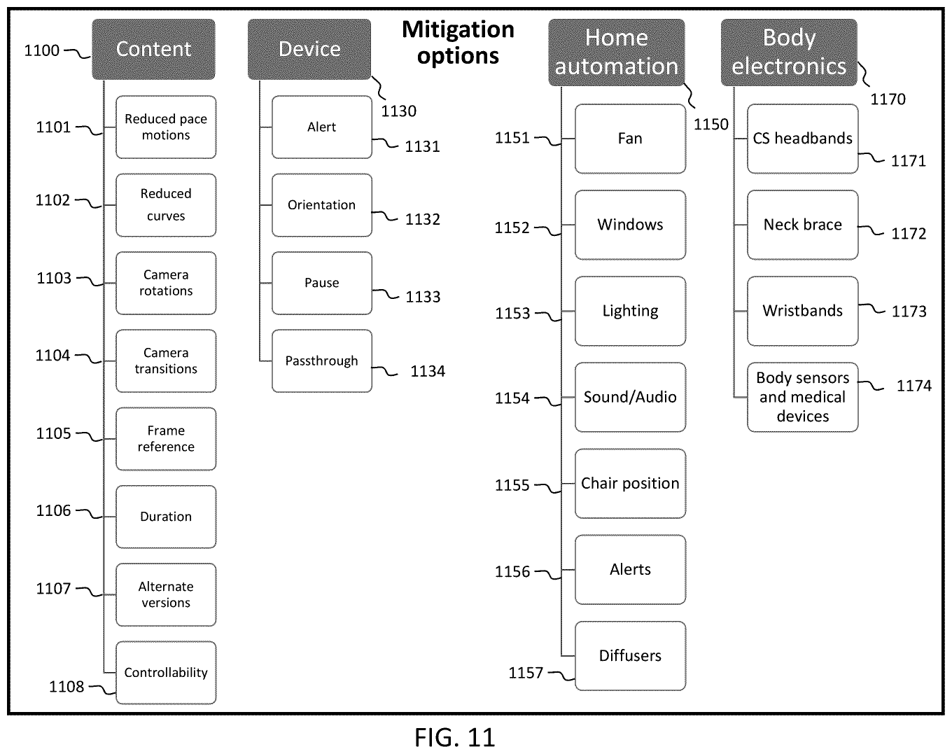

FIG. 11 is block diagram of mitigation options, in accordance with some embodiments of the disclosure. In some embodiments, mitigation options, which may be divided into mitigation categories and subcategories, may include altering the content 1100, determining device configurations 1130, performing home automation functions 1150, or performing functions on the user using body electronics 1170.

In some embodiments, as depicted at block 1100, mitigations that involve content alteration or skipping may be performed. Such mitigations may include slowing the pace of motions 1101, curve reduction 1102, camera rotations 1103, camera transitions 1104, frame reference 1105, duration 1106, alternate versions 1107, and controllability 1108.

As depicted in block 1101, in one embodiment, control circuitries 220 and/or 228 may provide content alterations, in response to determining that cybersickness above a threshold has occurred or is likely to occur, by slowing the pace of motions in the video content. In this embodiment, the control circuitry may reduce velocity, acceleration, rotations and other motions that are causative factors of cybersickness. In other embodiments, the content may be the same but the rate of transfer from the user's motions to the result in the content may be slowed down. As depicted earlier in FIG. 10, the velocity transfer function may be used by the control circuitry to transfer from current velocity of acceleration to a reduced velocity of acceleration when cybersickness severity above a predetermined level is determined. For example, the user may physically rotate their hand or press a game control button in the real world at a certain velocity and acceleration. However, the same velocity and acceleration as executed in the real-world may not be translated into the virtual game. Instead the system may only provide a 50 or 75% of effect in the virtual world. Continuing with this example, if the user performs a maneuver at a velocity of 20 MPH in the real world that is intended for a automobile maneuver in the virtual world, instead of transferring 100% of the velocity to the virtual world, the velocity transfer function may reduce the velocity to 15 MPH and have the automobile in the virtual world perform at that reduced velocity. By reducing the motions, velocity, and accelerations, the resultant effect on the user may reduce the cybersickness experienced by the user.

As depicted in block 1102, in one embodiment, control circuitries 220 and/or 228 may provide content alterations in response to determining that cybersickness above a threshold has occurred or is likely to occur by performing curve reductions in the video content. For example, if the video or XR reality experience is a car ride or roller coaster ride with a certain number of curves, the control circuitry may reduce the overall number of curves to mitigate cybersickness. If a determination is made that after the first few curves the user's biomarkers indicate a rise in cybersickness scores, then additional curves in the video content may be reduced or eliminated.

As depicted in block 1103, in one embodiment, control circuitries 220 and/or 228 may provide content alterations in response to determining that cybersickness above a threshold has occurred or is likely to occur by reducing camera rotations in the video content. For example, on a roller coaster ride, the camera may provide a feeling of a rotation several times to give a user a full 360° rotation as they would experience on a real-world roller coaster. In this embodiment, if the control circuitry determines that the current biomarkers indicate a rise in cybersickness, then the control circuitry may reduce or eliminate search camera rotations. Likewise, camera transitions 1104, in other directions outside of orientation may also be reduced when control circuitry determines that the current biomarkers indicate a rise in cybersickness.

As depicted in block 1105, in one embodiment, control circuitries 220 and/or 228 may provide content alterations in response to determining that cybersickness above a threshold has occurred or is likely to occur by changing the frame of reference for the user such that the video content is visualized from a different frame of reference. For example, if the current frame of reference has the user looking at an upside-down image while accelerating through a space, the control circuitry may use a different frame of reference such that the user is no longer upside-down and can visualize the movement at an angle that may not cause cybersickness or may reduce the amount of cybersickness.

As depicted in block 1106, in one embodiment, control circuitries 220 and/or 228 may provide content alterations in response to determining that cybersickness above a threshold has occurred or is likely to occur by reducing the duration of the video content. If the control circuitry determines that the current biomarkers indicate a rise in cybersickness, then the control circuitry may reduce the duration of a portion of the video that may be associated with the cybersickness. For example, if the video imagery is related to virtual video engagement where the user experiences bungee jumping from a really high altitude, driving a car at a high speed on winding roads, or riding on a portion of a roller coaster ride that has a lot of twists and turns, then the control circuitry may reduce the overall timing of such portion by a predetermined amount such that cybersickness is reduced or minimized.

As depicted in block 1107, in one embodiment, control circuitries 220 and/or 228 may provide content alterations in response to determining that cybersickness above a threshold has occurred or is likely to occur by using an alternate version of the portion of the video segment that has been determined to be the cause of the cybersickness. In this embodiment, the content creator may have created few different variations of a portion of the video segment that may be a leading cause of cybersickness. If such cybersickness is detected, the control circuitry may automatically switch from a current version to an alternate version that does not cause cybersickness.

As depicted in block 1108, in one embodiment, control circuitries 220 and/or 228 may provide content alterations in response to determining that cybersickness above a threshold has occurred or is likely to occur by providing controllability options in the video content. In this embodiment, the control circuitry may provide options to the user to control the speed on the curve, the rotation of a rollercoaster, or other controllable functions through the controller such that the user can control the speed, acceleration, and other factors to their liking so that cybersickness is not experienced or is reduced.

As depicted in block 1130, in one embodiment, control circuitries 220 and/or 228 may perform configurations in response to determining that cybersickness above a threshold has occurred or is likely to occur. In one embodiment, as depicted at block 1131, the device configuration may be to alert the user. In this embodiment, the control circuitry may provide an alert to the user while the user is viewing, consuming, or engaging with the video. The alert may notify the user that the current or upcoming segment of the video contains imagery that is likely to cause cybersickness. In addition to the alert, the device may also provide an option with the alert to skip the content or to switch to a pass-through mode where the user can visualize the real world instead of the virtual world that may cause cybersickness.

As depicted in block 1132, in another embodiment, the device configuration may be to orient the device to a different configuration. For example, the user may be instructed to rotate the device 90° such that the content is viewed in a different frame of reference or on a larger width to reduce cybersickness.

As depicted in block 1133, in another embodiment, as depicted at block 1131, the device configuration may be to pause the video. For example, the XR device may automatically pause the video for a predetermined duration, thereby allowing the user to regain their composure and reduce the effect of cybersickness. Likewise, in circumstances where the pause is not sufficient, the control circuitry may switch the camera of the XR device to a pass-through option 1134, where the user can view the real world around them and no longer be able to visualize any video or virtual content displayed on the XR device.

As depicted in block 1150, in one embodiment, control circuitries 220 and/or 228 may perform home automation functions in response to determining that cybersickness above a threshold has occurred or is likely to occur.

In another embodiment, if the control circuitries 220 or 228 determines that the current user is experiencing or is likely to experience cybersickness beyond a predetermined threshold, such as at blocks 603 or 609 of FIG. 6, then the control circuitries 220 or 228 may send a signal to lower the temperature, such as by turning on air conditioning (not shown in figure), or turning on the fan 1151 or increasing its speed, thereby allowing flow of air and lowering of temperature as a mitigation effort to reduce cybersickness. In some environments, the control circuitries 220 or 228 may also store the user's preferred temperature settings or fan speed and automatically set the thermostat to reach the preferred temperature either when cybersickness is experienced or in anticipation of the cybersickness.

In one embodiment, as depicted at block 1152, windows of a physical home may be controlled via an API as a mitigation option to reduce cybersickness. In this embodiment, the windows may include an automatic mechanism that allows the windows to be controlled electronically such that they can be opened and closed based on the type of signal received. In this embodiment, the windows may be connected wirelessly to either the control circuitries 220 or 228 associated with the headset or other intermediary home hub that is connected to both the headset and the automated windows.

In another embodiment where home automation is applied as a cybersickness mitigation option, as depicted at block 1153, lighting in the room in which the current user is viewing, consuming, or engaging with the video may be changed to reduce cybersickness. In this embodiment, the lighting modules and controllers may include a Wi-Fi or API capability that allows the lighting modules and controllers to be controlled electronically such that the light can be turned on/off or the intensity of the lighting can be changed.

For example, Smart Home lightning controllers, switches, and modules, such as smart switches from Lutron™, Insteon™, Wemo™, and Phillips™, which may include Wi-Fi controlling capability, may be accessed by the control circuitry. In this embodiment, the smart lighting switches and smart home devices with lighting may be connected wirelessly to either the control circuitries 220 or 228 associated with the headset or to a cybersickness server.

In one embodiment, if the control circuitries 220 or 228 determines that the user has experienced cybersickness, then the control circuitries 220 or 228 may send a signal to the smart lighting switch. The signal may turn on the lighting in the user's location, brighten the lighting, or, in case of lights that are movable, move the lighting direction to point in the direction of the user, thereby illuminating the user's area in an attempt to allow the user's brain to react positively to brighter lights and reduce the user's cybersickness. Since poor peripheral vision or lack of awareness of the surroundings can be contributing factors for cybersickness, such lighting may help in reducing the cybersickness.

In one embodiment, if the control circuitries 220 or 228 determines that the user has experienced cybersickness, then the control circuitries 220 or 228 may send a signal to a speaker or a device that includes a speaker to produce soothing sounds 1154, such as soothing and calming music to reduce cybersickness for the user. In another embodiment, if the control circuitries 220 or 228 determines that the user has experienced cybersickness, then the control circuitries 220 or 228 may mute the audio of the displayed content, lower its volume, or switch to calming music within the app.

In another embodiment, if the control circuitries 220 or 228 determines that the user has experienced cybersickness, then the control circuitries 220 or 228 may automatically straighten, recline, or adjust the angle of the chair 1155 in which the user is sitting and viewing, consuming or engaging with the video content. Since the wrong posture may also be a contributing factor to cybersickness, changing the angles of the chair, whether making it upright or at a different angle, may be used as a mitigation option for reducing cybersickness.

Other home automation options, such as alerting 1156 the user using speakers in the vicinity or automatically activating diffusers 1157 with certain incense or soothing smells or chemicals may also be performed.

As depicted in block 1157, in one embodiment, control circuitries 220 and/or 228 may activate certain body electronics in response to determining that cybersickness above a threshold has occurred or is likely to occur. Such body electronics may include cybersickness headbands 1171, neck braces 1172, wristbands 1173, or body sensors and medical devices 1174. In this embodiment, the control circuitry may use headbands 1171, neck braces 1172, wristbands 1173, or body sensors and medical devices 1174 and electronics worn by the user to send vibrations that fool the brain into disregarding the motion signals that may cause cybersickness in the user. Basically, this is a method of distracting the user with other motions. When such distractions are performed, the human brain may stops processing certain sensations, such as those sensations from the video that may cause cybersickness, and pay attention to the vibrations instead. Such vibrations may target the vestibulocochlear.

Additional mitigation options that align the user movements in the real world with imagery in the virtual world may also be performed. Since matching user movements with the virtual imagery in the virtual world with minimal lag is important in reducing cybersickness among users, the mitigation option may be to closely follow user movements in the virtual world. This is because when the brain senses movement by getting signals from the inner ears, eyes, muscles, and joints and the movements are not accompanied with visual imagery matching the movements, among other factors, this may trigger cybersickness. This may be applicable in several scenarios, including a scenario in which the user performs hand movements in the virtual world such that an avatar of the user in the virtual world can emulate the same movement. In such instances, matching the movements at high bitrates may help reduce cybersickness.

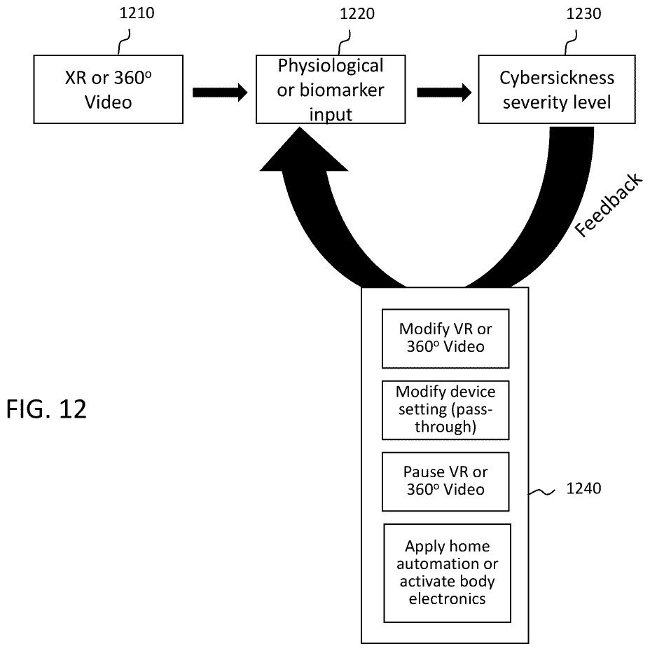

FIG. 12 is a block diagram of a feedback loop for mitigating cybersickness, in accordance with some embodiments of the disclosure. In this embodiment, the control circuitries 220 and/or 228 may use a real-time feedback loop to modify the display of content (e.g., skip or alter segments, etc.) or take other actions, such as changing device configurations, performing home automation, or activating electronics worn on the body, as described in relation to FIG. 11, to reduce cybersickness.

In one embodiment, the control circuitry may apply a certain cybersickness mitigation option and then determine whether application of that mitigation option resulted in a desired outcome, such as reducing the cybersickness severity level of the user. Since the mitigation option that may work for one user may not work for another user based on their background, demographics, medical history, and other factors, the control circuitry may apply the mitigation option to the current user and perform the feedback to determine whether a different mitigation option needs to be applied.

In one embodiment, a feedback loop may be deployed to obtain real-time biomarker data of the user after a mitigation option has been applied. Such feedback loop may determine whether a mitigation option that was applied achieved an anticipated result. If the mitigation option did not achieve an anticipated result, then such data may be fed back into the system to try additional mitigation approaches until the cybersickness severity level has gone down. In some embodiments, the feedback loop may be used to train the machine learning model for continued enhancement. The data may be used for subsequent users to predict cybersickness. In other embodiments, several iterations of mitigation options may be applied, each after receiving feedback as to whether an earlier mitigation option achieved the desired result. For example, the control circuitry may use an iterative approach by reducing velocity in a game to determine the effect. If the reduction in velocity reduces the cybersickness scores of users, but additional reduction is needed, then the control circuitry may further decrease the velocity using this iterative approach. If the reduction in velocity did not decrease the cybersickness score, then the control circuitry may apply a different mitigation option in an effort to reduce the cybersickness score.

As depicted at block 1210, in one embodiment, a video or extended reality experience may be viewed/consumed/engaged with by the current user. At block 1220, physiological or biomarker inputs are obtained during such viewing, consumption, or engagement with the video or extended reality experience. At block 1230, these inputs may be used by the control circuitry to determine a cybersickness severity level.

Upon determining that the cybersickness severity level exceeds a predetermined threshold, mitigation options at block 1240 may be applied. As discussed in further detail in the description related to FIG. 11, the mitigation options may include modifying or altering the video, modifying device settings, pausing the application, switching the camera on the XR device to a pass through such that the user can visualize the real world around them, or applying home automation or activating body electronics.

Once a mitigation option is selected and applied at block 1240, the system may again obtain physiological or biomarker inputs in real time at block 1220 to analyze the effect of applying the mitigation option. In some embodiments, the mitigation option may be sufficient to reduce the cybersickness score; in other embodiments the mitigation option may be a step in the right direction, but an additional application of the same mitigation option may be necessary to further reduce the cybersickness score, or the mitigation option may not be effective at all. Accordingly, the control circuitry may adjust and either apply the same or another mitigation option based on the feedback received. Applying such feedback loops may allow the control circuitry to use the data to enhance the predictability of cybersickness as well as determining its severity and the leading causes and determine which mitigation option works for the current user.

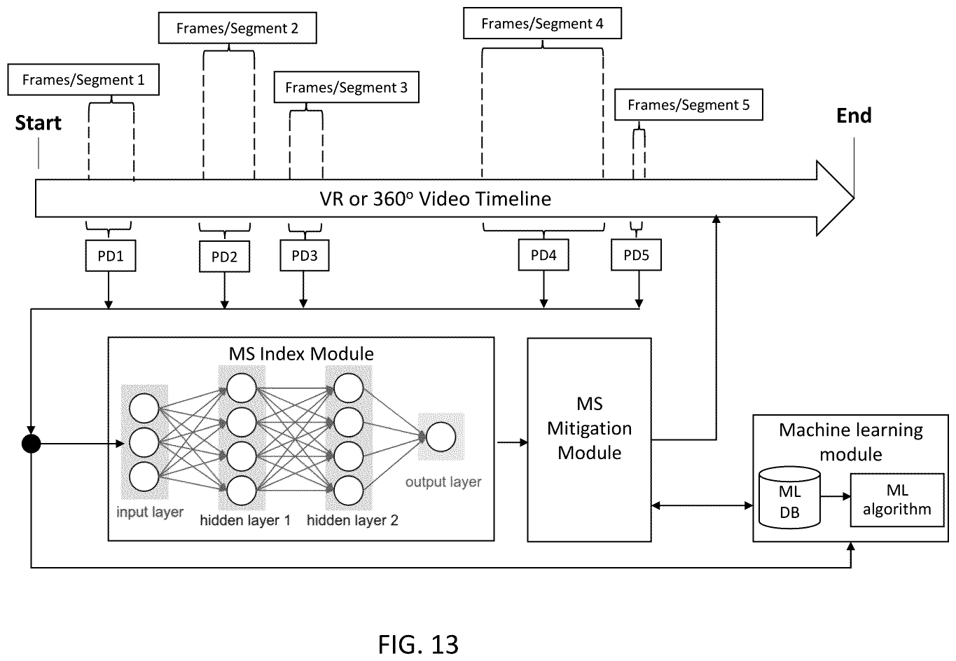

FIG. 13 is a block diagram of a feedback loop applied to selected segments and groups of frames for mitigating cybersickness, in accordance with some embodiments of the disclosure.

In one embodiment, the feedback loop is applied to the current user's biomarker data by applying a cybersickness mitigation option and determining the result of such mitigation option to further determine whether and which subsequent mitigation options, if any, need to be applied. In this embodiment, the control circuitries 220 and/or 228 may during their first pass determine that a group of frames, or a segment or portion of the video, such as segments 1, 2, 3, 4, and 5, are associated with content that has caused cybersickness in the current user. The control circuitries 220 and/or 228 may obtain physiological data PD1-PD5 that are associated with the groups of frames, or segments 1, 2, 3, 4, and 5, of the video that caused cybersickness.

In one embodiment, the control circuitries 220 and/or 228 may then use that physiological data as an input layer to determine a CSS score. Once the CSS score is determined, the control circuitries 220 and/or 228 may select a mitigation option from the mitigation module and apply it to the various segments 1, 2, 3, 4, and 5 based on the CSS score associated with those segments. For example, PD1 may be associated with a CSS score that relates to a moderate severity level, while PD4 may be associated with CSS score that relates to a high severity level. Accordingly, mitigation options that apply to each severity level may be applied.

In one embodiment, once a mitigation option has been applied to the various segments, the control circuitry may once again obtain physiological data PD1-PD5 that is associated with the groups of frames, or segments 1, 2, 3, 4, and 5, to determine if a change in physiological data has occurred after the application of the mitigation option. In this second pass, the control circuitry may determine that the cybersickness score associated the group of frames, or a segment or portion of the video, such as segments 1, 2, 3, 4, and 5, was reduced by 15% based on the application of the mitigation option. If a desired predetermined result was to reduce the cybersickness score by 25%, determining that the prior mitigation option reduced the score somewhat, the control circuitry may once again apply the same mitigation option to further reduce the cybersickness score. As such, in some embodiments, the control circuitry may perform few iterations of the same mitigation option based on the feedback received until the cybersickness score has reduced by 25%. In another embodiment, the control circuitry may determine that another mitigation option may result in a faster reduction of the cybersickness score and as such may apply the other mitigation option. The control circuitry may also determine that the mitigation option applied in the first pass did not result in any reduction in cybersickness score, and therefore use a different mitigation option in an attempt to reduce the cybersickness score. The control circuitries 220 and/or 228 may also add additional hidden layers of data, such as the layers of data described in the description of FIG. 8, to refine their approach and select a different mitigation option based on the user's background or medical data that may have a greater effect on reducing cybersickness.

In another embodiment, the feedback loop is applied to data from a plurality of users' physiological data. In this embodiment, physiological data from a plurality of users who have consumed, viewed, or engaged with the same video or XR experience may be used. In this embodiment, the control circuitry may obtain the physiological data and categorize the plurality of users into different groups based on their background, medical history, demographics, and other data. The control circuitry then determines if the current user matches any characteristic with the background, medical history, demographics, and other data collected for the group.

Based on the matches, the control circuitry may apply similar mitigation options that were applied to the plurality of users with whom the characteristic was matched and determines whether the applied mitigation option results in a desired reduction of cybersickness score for the current user.

For example, the calculations based on physiological data for one group of users (Group 1) who are between the ages of 25-35 results in a CSS score of 75. In this example, for Group 1, application of mitigation option 1 reduced their CSS score to 60. The calculations based on physiological data for a second group of users (Group 2) who are between the ages of 45-55 also results in a CSS score of 75. In this example, for Group 2, application of mitigation option 2 reduced their CSS score to 60. If the current user's profile indicates that their age is 50, then, because the characteristic of age of shared with Group 2, the control circuitry may apply mitigation option 2, rather than mitigation option 1, in an effort to reduce the current user's cybersickness score.

Once a mitigation option is selected and applied based on the shared characteristic with the group of users in Group 2 (not shown in figure), the control circuitry may again obtain physiological or biomarker inputs in real time to analyze the effect of applying the mitigation option.

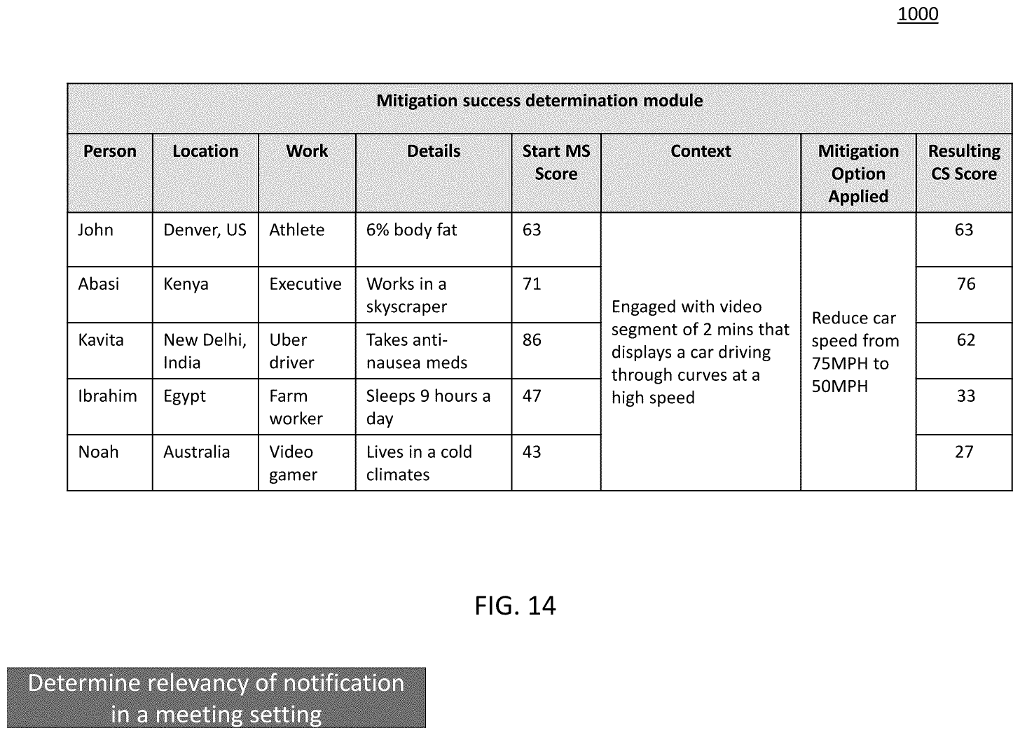

FIG. 14 is a table of different CSS scores of different users based on a same mitigation option applied based on the severity of cybersickness it can cause, in accordance with some embodiments of the disclosure. In this example, location, type of work, and other details may factor in for determining which mitigation option may result in a better reduction in cybersickness score. For example, as depicted, Johnny works in Denver, Colorado, United States. Because Denver is located at the altitude that is a mile above sea level, the data related to Johnny's cybersickness may not be applicable to other users who live at sea level or below sea level locations. Likewise, Abasi lives in Kenya and works in a skyscraper. The biomarker or physiological data relating to cybersickness for Abasi may be specific to climatic conditions in Kenya and for users who may be used to movements in the floor because they work in moving environments such as skyscrapers, boats, or airplanes. Biomarker or physiological data relating to cybersickness for Kavita, who is an Uber driver and used to being constantly in motion, may also be applicable to other users who are also in motion for long durations. Biomarker or physiological data relating to cybersickness for Ibrahim, who lives on a farm and may not be exposed to pollution and gets nine hours of sleep a night and is well rested, may be applicable to other users who also get plenty of rest and live in a clean air environment. Biomarker or physiological data relating to cybersickness for Noah, who is a video gamer and thoroughly used to virtual reality experiences, may be applicable to those users who are also gamers or used to longer durations of experiencing virtual reality and have a higher tolerance for cybersickness.

As depicted, the same mitigation option, which is to reduce car speed from 75 MPH to 25 MPH, is applied as a mitigation option to all the users. The mitigation option is applied in response to determining that two minutes of the video include driving of a car through curves at high speed. Since each user has a different background, location, and tolerance to cybersickness, applying the same mitigation option to all of them results in a different cybersickness score for each user. The system may determine that in order to effectively address cybersickness for each of the individuals, their background, location, sex, medical data and other layers of information, such as layers of information described in relation to FIG. 8, may need to be factored in in order to customize the choice of mitigation option.

Since each mitigation option may have different outcomes, which may be specific to a user based on their background, demographics, and other layers of information, the control circuitry may select between two mitigation options based on a mitigation option that achieves the best results, or at least reduces the cybersickness score below the predetermined threshold severity level, for a demographic group to which the user belongs. In other words, if a first mitigation option results in a higher reduction of the cybersickness score for members of a demographic group to which the user belongs than a second mitigation option, then the first mitigation option may be selected and may be automatically executed by the control circuitry.

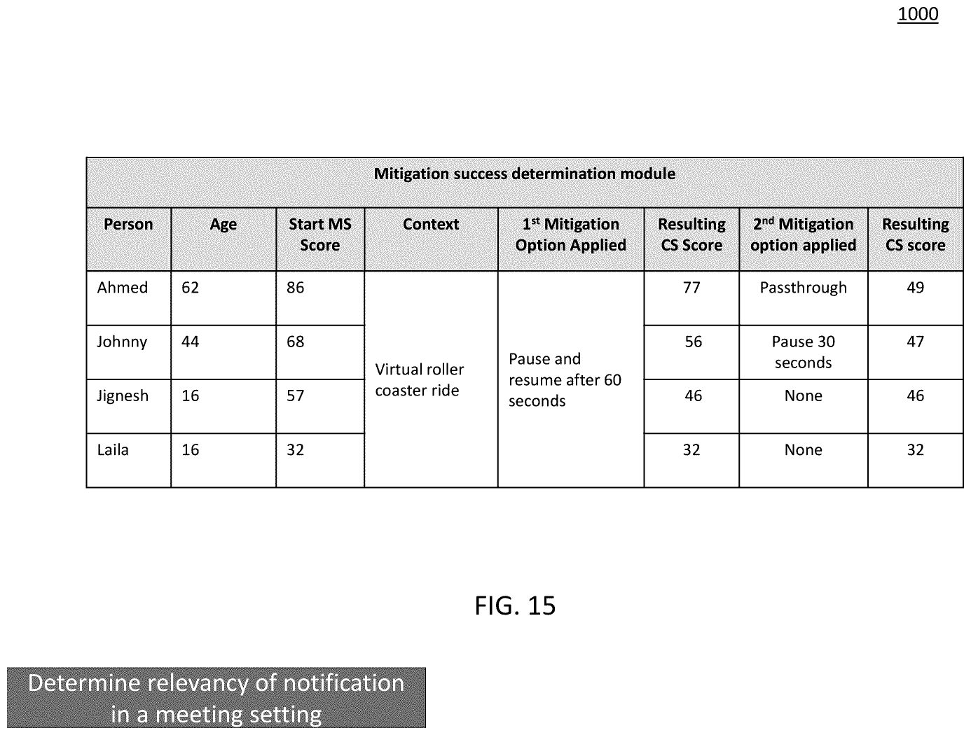

FIG. 15 is an example of a series of mitigation options applied to a plurality of users based on the feedback received, in accordance with some embodiments of the disclosure. In this embodiment, the control circuitry may determine that applying a first mitigation option that is the same for all the users may not result in a reduction of cybersickness scores as desired for all users. As such, the control circuitry may take into account different layers of information that are specific to each user and apply a second mitigation option that might be different for each user and then reassess in real time whether the cybersickness scores have been reduced. For example, a second mitigation option, a pass-through, was applied for Ahmed, whereas pausing for 30 seconds, as a second mitigation option, was applied for Johnny, each based on their backgrounds and layers of information.

In some embodiments, the control circuitry may determine the success of the mitigation option and select the mitigation option that provides the highest results, such as most reduction in cybersickness score or the fastest reduction in cybersickness score. As depicted in the table, an inference can be drawn that a same mitigation option applied across the board to all users may not result in the best outcome. As such, in determining which mitigation option may result in the best success or outcome, the layers of information, such as age and other factors, may be considered by the control circuitry.

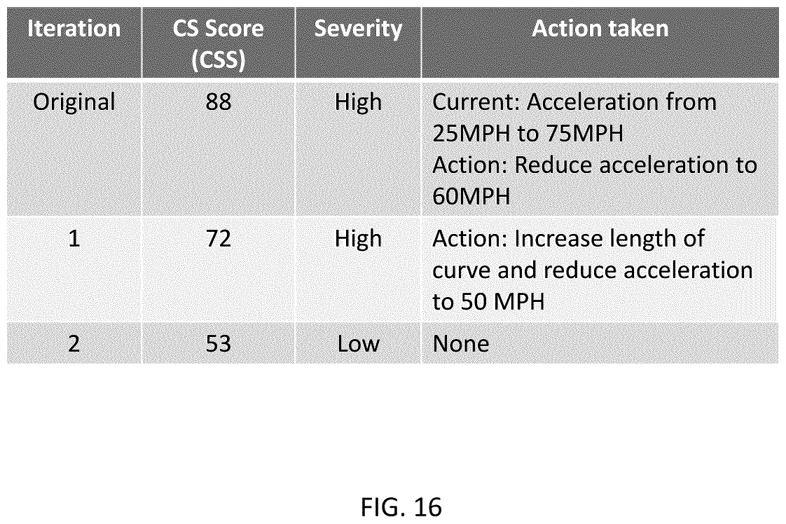

FIG. 16 is a table depicting the results of iterative mitigation options applied to one user based on feedback to reduce the cybersickness score, in accordance with some embodiments of the disclosure. In this embodiment, the control circuitry may calculate an initial CSS score based on the physiological or biomarker data obtained for a current user. Based on the calculated CSS score of 88, the control circuitry may associate a high severity level with the CSS score. Accordingly, the control circuitry may apply a mitigation option to reduce the top speed to 60 MPH (i.e., going from 25 MPH to 60 MPH) from the original acceleration, which was going from 25 MPH to 75 MPH.

After the mitigation option has been applied, the control circuitry may obtain physiological data in real time to determine whether the applied mitigation option resulted in a reduction of the CSS score. As depicted, based on the mitigation option applied, the CSS score dropped to 72 in the first iteration. The control circuitry may apply a different mitigation option of altering the video content by increasing a length of a curve, thereby attempting to reduce the sharpness of the curve, and also apply the reducing the acceleration mitigation option again, reducing the acceleration from 25 MPH to 50 MPH. After this second iteration of applying the mitigation options, which was based on feedback received from the first iteration, the control circuitry may once again obtain physiological data in real time to determine whether the applied mitigation option during the second iteration resulted in a reduction of CSS score. As depicted, the resulting CSS score was determined to be 53, which may be associated with a low sickness severity. Since the low CSS score may be acceptable, the control circuitry may end the process of iteratively applying additional mitigation options.

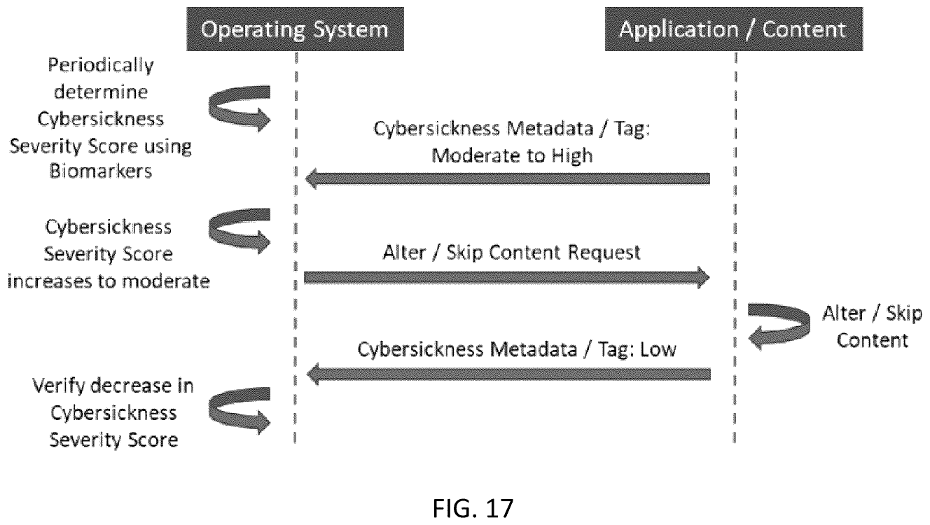

FIG. 17 is an example of a communication process between an operating system and application or content module for mitigating cybersickness, in accordance with some embodiments of the disclosure. In this embodiment, the operating system may communicate with the application/content module to reduce the cybersickness severity level from high to low.

As depicted, the operating system may periodically determine cybersickness severity scores using biomarkers. It may also receive cybersickness metadata from the application and content module. Based on the data received, the operating system may determine that the cybersickness severity score has increased to a moderate level. Accordingly, the operating system may send a message to the application/content module to alter and skip content in order to reduce the moderate cybersickness severity. The application/content module may perform the alerting and skipping of content and provide resultant cybersickness metadata to the operating system after such operation has been completed. The operating system receives the metadata and again determines the cybersickness score. In this example, the operating system may determine that after the altering and skipping was performed, the cybersickness score was reduced to a low severity level.

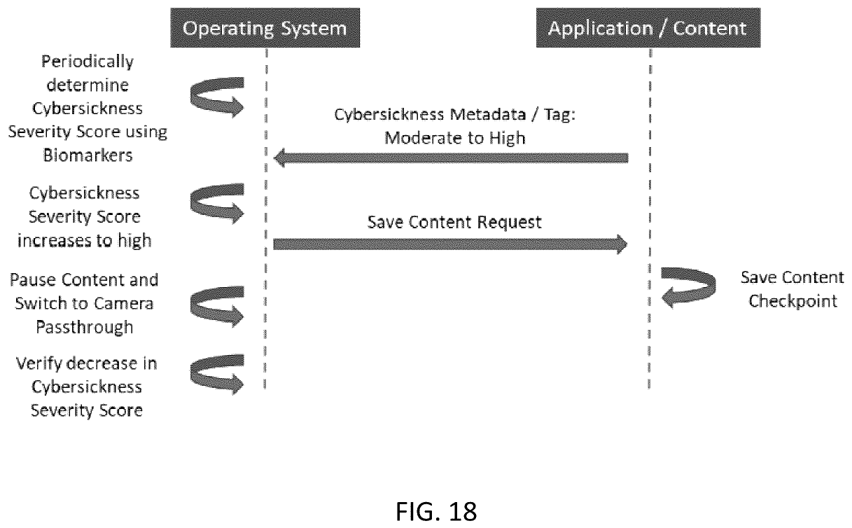

FIG. 18 is another example of a communication process between an operating system and application or content module for mitigating cybersickness, in accordance with some embodiments of the disclosure. Similar to FIG. 17, the operating system may apply different mitigation options and determine whether such applications reduce the cybersickness score. The operating system may communicate with the application/content module and receive metadata in response to applying a mitigation option, which, in FIG. 18, is pausing content and switching the camera to pass-through. The operating system may instruct the application/content module to save the content checkpoint such that once a user returns to the content it can be picked up from the same checkpoint prior to the operating system pausing and switching to pass-through camera mode.

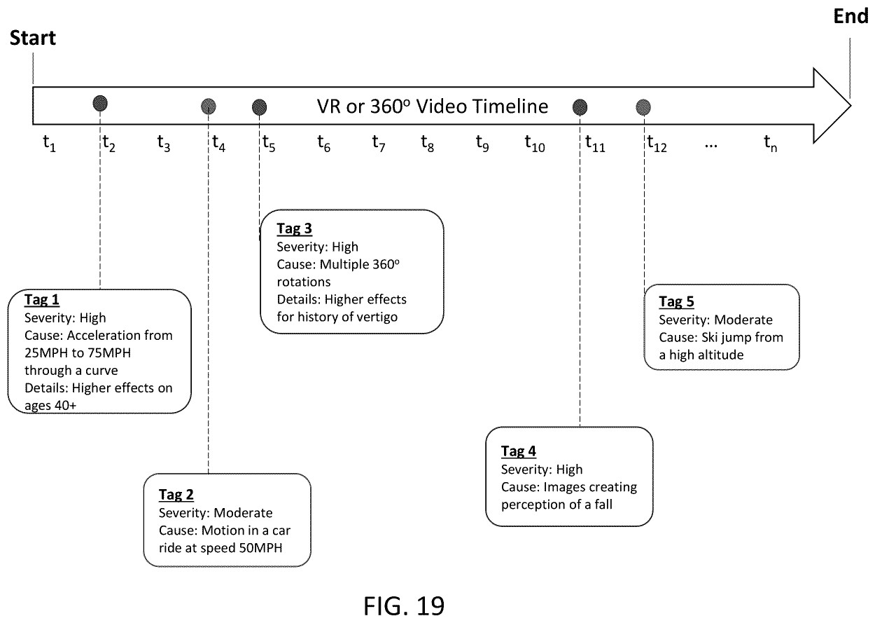

FIG. 19 is a block diagram of a plurality of metadata tags applied to content that has been determined to cause cybersickness, in accordance with some embodiments of the disclosure. In some embodiments, the control circuitry may tag portions of the video content that caused cybersickness. Such tagging may allow the control circuitry to subsequently alert the same user or other users when they are approaching the portion of content that caused cybersickness by alerting them to expect cybersickness during the tagged segment.

The tags may provide a variety of information including the severity of cybersickness the segment has caused, the cause of cybersickness, and any other details such as the effect of cybersickness on certain categories of users. For example, as depicted, Tag 1 may indicate that a high cybersickness severity level is to be expected from the content tagged by Tag 1. The tag may also indicate that the cause of cybersickness is due to an acceleration from 25 MPH to 75 MPH through a curve that is depicted in the virtual experience. The tag may also indicate that the content portion has a higher effect on users who are of age 40-plus.

The tags may also be color-coded or have some distinguishing pattern, highlighting, or icon to distinguish a tag associated with moderate severity level from a tag associated with a high severity level. The tags may also include timestamps of the start and end of the group of frames, segment, or portion of the video with which the high cybersickness score is associated.

It will be apparent to those of ordinary skill in the art that methods involved in the above-described embodiments may be embodied in a computer program product that includes a computer-usable and/or -readable medium. For example, such a computer-usable medium may consist of a read-only memory device, such as a CD-ROM disk or conventional ROM device, or a random-access memory, such as a hard drive device or a computer diskette, having a computer-readable program code stored thereon. It should also be understood that methods, techniques, and processes involved in the present disclosure may be executed using processing circuitry.

The processes discussed above are intended to be illustrative and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

1. A method comprising:
obtaining a plurality of biometric data measurements for a user wearing an extended reality (XR) device during a portion of playing of a content item played using the XR device;
determining a cybersickness score based on the plurality of biometric data measurements;
in response to determining that the cyber sickness score exceeds a severity threshold:
automatically executing a remedial action selected based on the cybersickness score to mitigate the cybersickness; and
storing a metadata tag in association with the portion of the content item.

2. The method of claim 1, wherein the remedial action includes altering or skipping the portion of the content item.

3. (canceled)

4. The method of claim 1, wherein the remedial action includes switching a camera of the XR device to a pass-through mode that allows the real-world environment to be viewed via the XR device.

5. The method of claim 1, further comprising:
monitoring biometric data measurements of the user during playing of the content item;
determining that the cybersickness score during a plurality of portions of the XR content exceeds the severity threshold; and
tagging each of the plurality of portions of the XR content during which the cybersickness score exceeds the severity threshold with a metadata tag that identifies the severity level.

6. The method of claim 5, further comprising:
determining that the same content item is displayed on an XR device of a second user;
determining that a portion of the XR content, from the plurality of portions that are tagged, is being displayed on the second user's XR device; and
in response to such determination:
automatically executing a remedial action selected based on the severity level of the tagged portion of content that is being displayed.

7. The method of claim 6, wherein the remedial action is transmitting an alert to the second user's XR device, wherein the alert includes information relating to the severity level of the tagged portion of content that is being displayed.

8. (canceled)

9. The method of claim 1, further comprising:
determining that the cybersickness score relates to a highest level of severity; and
in response to the determination, switching a camera of the XR device to pass-through mode such that a real-world environment can be viewed through the XR device.

10. The method of claim 1, further comprising:
obtaining, after executing the remedial action, an updated plurality of biometric data measurements for the user wearing the XR device during the playing of the portion of the content item played using the XR device;
comparing the updated plurality of biometric data measurements with the plurality of biometric data measurements obtained prior to executing of the remedial action; and
based on the comparison, determining whether the cybersickness score was reduced based on the automatic execution of the remedial action.

11. The method of claim 10, further comprising:
in response to determining, based on the comparison, that the cybersickness score was not reduced after the automatic execution of the remedial action, executing a different type of remedial action than the remedial action previously executed.

12. (canceled)

13. (canceled)

14. The method of claim 1, wherein the remedial action selected based on the cybersickness score is to reduce velocity or acceleration of an action performed by the user in a real world when the velocity or acceleration is applied to the portion of the content in a virtual world.

15. (canceled)

16. A system comprising:
communications circuitry configured to access an extended reality (XR) device; and
control circuitry configured to:
obtain a plurality of biometric data measurements for a user wearing the extended reality (XR) device during a portion of playing of a content item played using the XR device;
measurements;
determine a cybersickness score based on the plurality of biometric data in response to determining that the cyber sickness score exceeds a severity threshold:
automatically execute a remedial action selected based on the cybersickness score to mitigate the cybersickness; and
store a metadata tag in association with the portion of the content item.

17. The system of claim 16, wherein the remedial action includes the control circuitry configured to alter or skip the portion of the content item.

18. (canceled)

19. The system of claim 16, wherein the remedial action includes the control circuitry configured to switch a camera of the XR device to a pass-through mode that allows the real-world environment to be viewed via the XR device.

20. The system of claim 16, further comprising, the control circuitry configured to:
monitor biometric data measurements of the user during playing of the content item;
determine that the cybersickness score during a plurality of portions of the XR content exceeds the severity threshold; and
tag each of the plurality of portions of the XR content during which the cybersickness score exceeds the severity threshold with a metadata tag that identifies the severity level.

21. The system of claim 20, further comprising, the control circuitry configured to:
determine that the same content item is displayed on an XR device of a second user;
determine that a portion of the XR content, from the plurality of portions that are tagged, is being displayed on the second user's XR device; and
in response to such determination:
automatically execute a remedial action selected based on the severity level of the tagged portion of content that is being displayed.

22. The system of claim 21, wherein the remedial action is transmitting an alert by the control circuitry to the second user's XR device, wherein the alert includes information relating to the severity level of the tagged portion of content that is being displayed.

23. (canceled)

24. The system of claim 16, further comprising, the control circuitry configured to:
determine that the cybersickness score relates to a highest level of severity; and
in response to the determination, switch a camera of the XR device to pass-through mode such that a real-world environment can be viewed through the XR device.

25. The system of claim 16, further comprising, the control circuitry configured to:
obtain, after executing the remedial action, an updated plurality of biometric data measurements for the user wearing the XR device during the playing of the portion of the content item played using the XR device;
compare the updated plurality of biometric data measurements with the plurality of biometric data measurements obtained prior to executing of the remedial action; and
based on the comparison, determine whether the cybersickness score was reduced based on the automatic execution of the remedial action.

26. The system of claim 25, further comprising, the control circuitry configured to:
in response to determining, based on the comparison, that the cybersickness score was not reduced after the automatic execution of the remedial action, execute a different type of remedial action than the remedial action previously executed.

27. (canceled)

28. (canceled)

29. The system of claim 16, wherein the remedial action selected by the control circuitry is based on the cybersickness score is to reduce velocity or acceleration of an action performed by the user in a real world when the velocity or acceleration is applied to the portion of the content in a virtual world.

30. (canceled)
